(19) United States
(12) Patent Application Publication
(10) Pub. No.: US 2025/0302761 A1
(43) Pub. Date: Oct. 2, 2025

(54) EXTRACELLULAR VESICLES FUNCTIONALIZED WITH AN ERV SYNCITIN AND USES THEREOF FOR CARGO DELIVERY

(71) Applicants: INSTITUT NATIONAL DE LA SANTÉ ET DE LA RECHERCHE MÉDICALE, PARIS (FR); UNIVERSITÉ PARIS CITÉ, PARIS (FR); UNIVERSITÉ PARIS SCIENCES ET LETTRES, PARIS (FR)

(72) Inventors: Grégory LAVIEU, PARIS (FR); Julie DANCOURT, PARIS (FR); Shéryl BUI, PARIS (FR)

(21) Appl. No.: 18/881,505

(22) PCT Filed: Jul. 20, 2023
(86) PCT No.: PCT/EP2023/070159

§ 371 Date: Jan. 6, 2025

(30) Foreign Application Priority Data
Jul. 21, 2022 (EP) 22306089.8

(51) Int. Cl. A61K 9/50; A61K 38/16; A61K 47/64; A61K 47/69; C12N 9/22; C12N 15/11

(52) U.S. Cl. CPC A61K 9/5068; A61K 38/164; A61K 47/64; A61K 47/6901; C12N 9/226; C12N 15/11; C12N 2310/20

(57) ABSTRACT

EVs are being recognized as vectors for drug delivery. In particular. EV loading with targeting and therapeutic agents brings along an interesting opportunity to translate EVs into a bio-mimetic selective delivery system. Indeed. EVs constitute a physiological carrier being potentially less immunogenic than artificial delivery vehicles. The inventors now developed a novel method to control the loading of a cargo into EVs on demand. These EVs are equipped, if necessary, with non-viral fusogen, therefore enhancing EV-cargo delivery into acceptor cells. To acutely measure this process, they follow the fate of a luciferase-tagged cargo. Cargo loading was enabled through a drug-reversible inducible dimerization system. Briefly, donor cells were transfected with plasmids encoding for FKBP-tagged CD63, a classical membrane EV marker, and FRB-Nanoluciferase (NLuc) that is normally cytosolic. Upon addition of the dimerizing drug. FRB-Nluc interacts with FKBP-CD63 and is recruited into secreted EVs. This is accompanied by an enhanced delivery into acceptor cells. This phenomenon can be further enhanced when EVs are equipped with syncitin1, a mammalian fusogenic protein that trigger fusion between EV membrane and the plasma membrane of acceptor cells. Using this novel process, the inventors further demonstrated that the catalytic domain of the Diphteria toxin (DTA), that is responsible for protein synthesis inhibition and ultimately cell death, can be delivered to acceptor cells via functionalized EVs. This led to protein synthesis inhibition and death of acceptor cells. This novel method and the derived applications promise to open new doors in precision care medicine, especially when EVs will be equipped with antibodies raised against cell specific antigens.

### FIELD OF THE INVENTION

The present invention is in the field of medicine, in particular in the field of cargo delivery into target cells.

### BACKGROUND OF THE INVENTION

Extracellular Vesicles (EVs) are now recognized as vectors of intercellular communication capable of transferring nucleotides, lipids, and proteins from donor to acceptor cells (Skog, J. et al. Glioblastoma microvesicles transport RNA and proteins that promote tumour growth and provide diagnostic biomarkers. Nat. Cell Biol. 10, 1470 1476 (2008); Valadi, H. et al. Exosome-mediated transfer of mRNAs and microRNAs is a novel mechanism of genetic exchange between cells. Nat. Cell Biol. 9, 654 659 (2007); Flaherty, S. E. et al. A lipase-independent pathway of lipid release and immune modulation by adipocytes. Science (80-.). 363, 989 993 (2019); Al-Nedawi, K. et al. Intercellular transfer of the oncogenic receptor EGFRvIll by microvesicles derived from tumour cells. Nat. Cell Biol. 10, 619 24 (2008)). EV-mediated communication has been associated with many physiological and pathophysiological functions, including cancer, immune responses, cardiovascular diseases, lipid homeostasis, regeneration and stem cell-based therapy (Mathieu, M., Martin-Jaular, L., Lavieu, G. & Théry, (. Specificities of secretion and uptake of exosomes and other extracellular vesicles for cell-to-cell communication. Nat. Cell Biol. 21, 9 17 (2019)). The spectrum of tissues/cells that are capable to release or capture EVs is broad and includes, neuronal cells, adipocytes, as well as immune cells.

EVs are therefore being recognized as vectors of major importance for physiology in general, and appears as promising candidates for translational applications such as targeted drug delivery. In particular, EV loading with targeting and therapeutic agents brings along an interesting opportunity to translate EVs into a bio-mimetic selective delivery system. Indeed, EVs constitute a physiological carrier being potentially less immunogenic than artificial delivery vehicles. EVs may advantageously change cargo pharmacokinetics, biodistribution and bioavailability by (i) protecting cargos, (ii) addressing them to the site of interest and (iii) facilitating membrane transport (Murphy, D. F. et al. Extracellular vesicle-based therapeutics: natural versus engineered targeting and trafficking. Exp. Mol. Med. 51, 32 (2019)). Eventually, using EVs or chemically-formulated EV mimetics to deliver therapeutics (including the gene editing toolbox) to specific cells within the body would revolutionize cell/gene therapy.

### SUMMARY OF THE INVENTION

The present invention is defined by the claims. In particular, the present invention relates to extracellular vesicles functionalized with an ERV syncytin and uses thereof for cargo delivery.

### DETAILED DESCRIPTION OF THE INVENTION

### Main Definitions:

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some embodiments, the polynucleotide comprises an mRNA. In other aspect, the mRNA is a synthetic mRNA. In some embodiments, the synthetic mRNA comprises at least one unnatural nucleobase. In some embodiments, all nucleobases of a certain class have been replaced with unnatural nucleobases (e.g., all uridines in a polynucleotide disclosed herein can be replaced with an unnatural nucleobase, e.g., 5-methoxyuridine). In some embodiments, the polynucleotide (e.g., a synthetic RNA or a synthetic DNA) comprises only natural nucleobases, i.e., A, C, T and G in the case of a synthetic DNA, or A, C, T, and U in the case of a synthetic RNA.

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “polynucleotide sequence encoding an amino acid sequence” includes all nucleotide sequences that are degenerate versions of each other and that encode the same amino acid sequence. The phrase “polynucleotide sequence that encodes a protein or a RNA” may also include introns to the extent that the nucleotide sequence encoding the protein may in some version contain an intron(s).

As used herein, the expression “derived from” refers to a process whereby a first component (e.g., a first polypeptide), or information from that first component, is used to isolate, derive or make a different second component (e.g., a second polypeptide that is different from the first one).

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99 or 100% of identity with the second amino acid sequence.

As used herein, the term “mutation” has its general meaning in the art and refers to a substitution, deletion or insertion. In particular, the term “substitution” means that a specific amino acid residue at a specific position is removed and another amino acid residue is inserted into the same position. Within the specification, the mutation are references according to the standard mutation nomenclature.

As used herein, the term “ERV syncytin” has its general meaning in the art and refers to highly fusogenic envelope glycoproteins from eutherian mammals, which belong to the family of Endogenous Retroviruses (ERVs). These proteins are encoded by genes, which display a preferential expression in placenta and induce syncytium formation when introduced into cultured cells (Cornelis G, Heidmann O, Degrelle S A, Vernochet C, Lavialle C, Letzelter C, et al (2013). Captured retroviral envelope syncytin gene associated with the unique placental structure of higher ruminants PNAS 110 (9): E828 E837.).

As used herein, the term “syncytin-1” or “SYN” has its general meaning in the art and refers to a protein found in humans and other primates that is encoded by the ERVW-1 gene (endogenous retrovirus group W envelope member 1). Syncytin-1 is a cell-cell fusion protein whose function is best characterized in placental development. The term is also known as Endogenous retrovirus group W member 1, Env-W, Envelope polyprotein gPr73, Enverin, HERV-7q Envelope protein, HERV-W envelope protein, HERV-W_7q21.2 provirus ancestral Env polyprotein and Syncytin. An exemplary amino acid sequence for syncytin-1 is represented by SEQ ID NO: 1. The signal peptide ranges from the amino acid residue at position 1 to the amino acid residue at position 20 in SEQ ID NO: 1. The extracellular domain of syncytin-1 ranges from the amino acid residue at position 21 to the amino acid residue at position 443 in SEQ ID NO: 1.

SEQ ID NO: 1 >sp|Q9UQF0|SYCY1 HUMAN Syncytin-1
OS = Homo sapiens OX = 9606 GN = ERVW-1 PE = 1
SV = 1 (the signal peptide is indicated in
italic)
MALPYHIFLFTVLLPSFTLTAPPPCRCMTSSSPYQEFLWRMQRPGNID
APSYRSLSKGTPTFTAHTHMPRNCYHSATLCMHANTHYWTGKMINPSC
PGGLGVTVCWTYFTQTGMSDGGGVQDQAREKHVKEVISQLTRVHGTSS
PYKGLDLSKLHETLRTHTRIVSLFNTTLTGLHEVSAQNPTNCWICLPL
NFRPYVSIPVPEQWNNFSTEINTTSVLVGPLVSNLEITHTSNLTCVKF
SNTTYTTNSQCIRWVTPPTQIVCLPSGIFFVCGTSAYRCLNGSSESMC
FLSFLVPPMTIYTEQDLYSYVISKPRNKRVPILPFVIGAGVLGALGTG
IGGITTSTQFYYKLSQELNGDMERVADSLVTLQDQLNSLAAVVLQNRR
ALDLLTAERGGTCLFLGEECCYYVNQSGIVTEKVKEIRDRIQRRAEEL
RNTGPWGLLSQWMPWILPFLGPLAAIILLLLFGPCIFNLLVNFVSSRI
EAVKLQMEPKMQSKTKIYRRPLDRPASPRSDVNDIKGTPPEEISAAQP
LLRPNSAGSS

As used herein, the term “ASCT1” refers to the human neutral amino acid transporter A that is encoded by the SLCIA4gene. Syncytin-1 can bind to ASCT1 (Antony J M, Ellestad K K, Hammond R, Imaizumi K, Mallet F, Warren K G, Power C. The human endogenous retrovirus envelope glycoprotein, syncytin-1, regulates neuroinflammation and its receptor expression in multiple sclerosis: a role for endoplasmic reticulum chaperones in astrocytes. J Immunol. 2007, Jul. 15; 179 (2): 1210-24. doi: 10.4049 jimmunol. 179.2. 1210. PMID: 17617614).

As used herein, the term “ASCT2” refers to the neutral amino acid transporter B (0) that is encoded by the SLC1A5 gene. ASCT2 was described as the receptor for syncytin-1 (Blond J L, Lavillette D, Cheynet V, Bouton O, Oriol G, Chapel-Fernandes S, Mandrand B, Mallet F, Cosset F L. An envelope glycoprotein of the human endogenous retrovirus HERV-W is expressed in the human placenta and fuses cells expressing the type D mammalian retrovirus receptor. J Virol. 2000; 74:3321-3329. doi: 10.1128 JVI.74.7.3321-3329.2000.).

As used herein, the term “syncitin-1 polypeptide” or “SYN polypeptide” refers to any polypeptide thar derives from syncytin-1 and that comprises the SDGGGX2DX2R (SEQ ID NO: 19) conserved motif essential for syncytin-1-hASCT2 interaction (see Cheynet V, Oriol G, Mallet F. Identification of the hASCT2-binding domain of the Env ERVWEI syncytin-1 fusogenic glycoprotein. Retrovirology. 2006 Jul. 4; 3:41. doi: 10.1186 1742-4690-3-41. PMID: 16820059; PMCID: PMC1524976.). According to the present invention, the syncytin-1 polypeptide is capable of binding to the ASCT1 receptor, preferably ASCT2 receptor as determined by any assay well known in the art (see e.g. Cheynet V. et al. supra).

As used herein, the term “extracellular vesicle” or “EV” has its general meaning in the art and refers to a cell-derived vesicle comprising a membrane that encloses an internal space. Extracellular vesicles comprise all membrane-bound vesicles that have a smaller diameter than the cell from which they are derived. Generally, extracellular vesicles range in diameter from 50 nm to 1000 nm, and can comprise various macromolecular cargo either within the internal space, displayed on the external surface of the extracellular vesicle, and/or spanning the membrane.

As used herein, the term “functionalized” refers to the fact that the EV of the present invention incorporates in its membrane a polypeptide of interest (e.g. the ERV syncytin of the present invention).

As used herein, the terms “isolated” “isolating” “purified” “purifying,” “enriched,” and “enriching,” as used herein with respect to cells, means that the EVs at some point in time were separated, purified, and capable of therapeutic use. “Highly purified,” “highly enriched,” and “highly isolated,” when used with respect to said extracellular vesicles, indicates that the cells of interest are at least about 70%, about 75%, about 80%, about 85% about 90% or more of the cells, about 95%, at least 99% pure, at least 99.5% pure, or at least 99.9% pure or more of the cells, and can preferably be about 95% or more of the EVs.

As used herein, the term “donor cell” means a cell that is suitable for the production of the EVs of the present invention.

As used herein, the term “target cell” means a cell with which fusion with a EV of the present invention is desired.

As used herein, the term “cargo” as used herein describes any molecule, e.g. nucleic acid, polypeptide, pharmaceutical, etc. with a desired biological activity and suitable solubility profile that is encapsidated into the virus EV.

As used herein, the term “load” refers to the introduction or insertion of a substance or object into or onto a EV of the present invention. As used herein, the term “loading” refers to introducing or inserting a substance or object into or onto the EV of the invention.

As used herein, the term “targeting moiety” refers to any molecule that binds specifically to a target.

As used herein, the term “antibody” refers to immunoglobulin molecules and immunologically active portions of immunoglobulin molecules, i.e., molecules that contain an antigen binding site that immunospecifically binds to an antigen. In natural antibodies of rodents and primates, two heavy chains are linked to each other by disulfide bonds, and each heavy chain is linked to a light chain by a disulfide bond. There are two types of light chains, lambda (l) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate in the antibody binding site, or influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. For the antibodies described hereafter, the CDRs have been determined using CDR finding algorithms from www.bioinf.org.uk—see the section entitled «How to identify the CDRs by looking at a sequence» within the Antibodies pages.

As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. “Fragments” comprise a portion of the intact antibody, generally the antigen binding site or variable region. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

As used herein, the term “single domain antibody”, “sdAb” or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

As used herein, the term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “specificity” refers to the ability of an antibody to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of an antibody to a target molecule (e.g. an epitope). The affinity of a binding protein is given by the dissociation constant Kd. For an antibody said Kd is defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a KD of about 10⁻⁷ M or less, such as about 10⁻⁸ M or less, such as about 10⁻⁹ M or less, about 10⁻¹⁰ M or less, or about 10^{-11 M} or even less.

As used herein, the term “subject”, “host”, “individual” or “patient” refers to a mammal, preferably a human being, male or female at any age that is in-need of a therapy.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

### Extracellular Vesicles of the Present Invention:

The first object of the present invention relates to an isolated extracellular vesicle functionalized with an ERV syncytin and loaded with one or more cargo(s) of interest and that optionally functionalized with a targeting moiety.

### ERV Syncytin:

ERVs syncytins according to the invention can be selected from human syncytins (e.g.: HERV-W and HERV-FRD), murine syncytins (e.g.: syncytin-A and syncytin-B), syncytin-Ory1, syncytin-Car1, syncytin-Rum1 or their functional orthologs (Cornelis G, Heidmann O), Degrelle S A, Vernochet (, Lavialle C, Letzelter (, et al (2013). Captured retroviral envelope syncytin gene associated with the unique placental structure of higher ruminants PNAS 110 (9): E828 E837; Dupressoir A, Marceau G, Vernochet C, Benit L, Kanellopoulos C, Sapin V et al (2005). Syncytin-A and syncytin-B, two fusogenic placenta-specific murine envelope genes of retroviral origin conserved in Muridae. Proceedings of the National Academy of Sciences of the United States of America 102:725-730).

By functional orthologs it is intended orthologs proteins encoded by orthologs genes and that exhibit fusogenic properties. Fusogenic properties may be assessed in fusion assays as described in Dupressoir A, Marceau G, Vernochet C, Benit L., Kanellopoulos C, Sapin V et al (2005). Syncytin-A and syncytin-B, two fusogenic placenta-specific murine envelope genes of retroviral origin conserved in Muridae. Proceedings of the National Academy of Sciences of the United States of America 102:725-730. Briefly, cells are transfected for example by using Lipofectamine (Invitrogen) and about 1-2 μg of DNA for 5×10⁵ cells or calcium phosphate precipitation (Invitrogen, 5-20 μg of DNA for 5×10⁵ cells). Plates are generally inspected for cell fusion 24-48 h after transfection. Syncytia can be visualized by using May-Grünwald and Giemsa staining (Sigma) and the fusion index calculated as [(N−S)/T]×100, where N is the number of nuclei in the syncytia, S is the number of syncytia, and T is the total number of nuclei counted.

Human syncytins encompasses HERV-W and HERV-FRD. Functional orthologs of these proteins can be found in Hominidae. HERV-W refers to a highly fusogenic membrane glycoprotein belonging to the family of Human Endogenous Retroviruses (HERVs). HERV-W is an envelope glycoprotein; it is also called Syncytin-1. It has the sequence indicated in Ensembl database, corresponding to Transcript ERVW-1-001, ENST00000493463. HERV-FRD also refers to a highly fusogenic membrane glycoprotein belonging to the family of Human Endogenous Retroviruses (HERVs). HERV-FRD is an envelope glycoprotein, also called Syncytin-2. It has the sequence indicated in Ensembl database, corresponding to Transcript ERVFRD-1, ENSG00000244476.

Murine syncytins encompasses murine syncytin-A (i.e.: Mus musculus syncytin-A, synA) and murine syncytin-B (i.e.: Mus musculus syncytin-B, synB). Functional orthologs of these proteins can be found in the Muridae family. Murine syncytin-A is encoded by the syncytin-A gene. Syncytin-A has the sequence indicated 1 in Ensembl database Syna ENSMUSG00000085957. Murine syncytin-B is encoded by the syncytin-B gene. Syncytin-B has the sequence indicated in Ensembl databaseSynb ENSMUSG00000047977.

The syncytin-Ory1 is encoded by the syncytin-Ory1 gene. Functional orthologs of syncytin-Ory1 can be found in the Leporidae family (typically rabbit and hare).

The syncytin-Car1 is encoded by the syncytin-Car1 gene. Functional orthologs of syncytin-Car1 can be found in carnivores mammals from the Laurasiatheria superorder (Cornelis et al., 2012; Lavialle et al., 2013).

The syncytin-Rum1 is encoded by the syncytin-Rum1 gene. Functional orthologs of syncytin Rum-1 can be found in ruminant mammals.

In some embodiments, the ERV syncytin according to the invention can be typically selected from the group consisting of HERV-W, HERV-FRD, syncytin-A, syncytin-B, syncytin-Ory1, syncytin-Car1 and syncytin-Rum1 and their functional orthologs; preferably the ERV syncytin is selected from the group consisting of HERV-W, HERV-FRD, murine syncytin-A and their functional orthologs, more preferably the ERV syncytin is selected from the group consisting of HERV-W, HERV-FRD and murine syncytin-A and even more preferably the ERV syncytin is HERV-W or HERV-FRD.

In some embodiments, the ERV syncytin is a synctin-1 polypeptide.

In some embodiments, the syncytin-1 polypeptide comprises the amino acid sequence as set forth in SEQ ID NO: 2 (SDGGGX2DX2R) and is capable to bind to the ASCT1 receptor, preferably to the ASCT2 receptor.

In some embodiments, the syncytin-1 polypeptide comprises the amino acid sequence as set forth in SEQ ID NO: 3 (SDGGGVQDQAR).

In some embodiments, the syncytin-1 polypeptide of the present invention comprises the amino acid sequence as set forth in SEQ ID NO: 3 (SDGGGVQDQAR) and comprises at least 15, 20, 25, 30, 35, 40, 45, 50, 100, 150, 200, 250, 300, 350, 400, or 450 consecutive amino acids of SEQ ID NO: 1.

In some embodiments, the syncintin-1 polypeptide of the present invention comprises an amino acid sequence having at 70% of identity with the amino acid sequence that ranges from the amino acid residue at position 21 to the amino acid residue at position 538 in SEQ ID NO: 1. In some embodiments, the syncintin-1 polypeptide of the present invention comprises the amino acid sequence that ranges from the amino acid residue at position 21 to the amino acid residue at position 538 in SEQ ID NO: 1 wherein the arginine residue (R) at position 393 and the phenylalanine residue (F) at position 399 are mutated for conferring immunosuppressive activity (Mangeney M, Renard M, Schlecht-Louf G, Bouallaga I, Heidmann O, Letzelter C, Richaud A, Ducos B, Heidmann T. Placental syncytins: Genetic disjunction between the fusogenic and immunosuppressive activity of retroviral envelope proteins. Proc Natl Acad Sci USA. 2007 Dec. 18; 104 (51): 20534-9. doi: 10.1073 pnas.0707873105. Epub 2007 Dec. 12. PMID: 18077339; PMCID: PMC2154466). In some embodiments, the syncintin-1 polypeptide of the present invention comprises the amino acid sequence that ranges from the amino acid residue at position 21 to the amino acid residue at position 538 in SEQ ID NO: 1 wherein the arginine residue (R) at position 393 is substituted by a glutamine residue (Q) and the phenylalanine residue (F) are position 399 is substituted by an alanine residue (A).

### Cargo:

Typically, the cargo can be of any nature compatible with the loading in EVs.

In some embodiments, the cargo is selected from the group consisting of organic molecules, polymers, polypeptides polynucleotides and small organic compounds having a molecular weight of more than 50 and less than about 2,500 daltons. Cargos are also found among biomolecules including peptides, saccharides, fatty acids, lipids, steroids, purines, pyrimidines, derivatives, structural analogs or combinations thereof.

In some embodiments, cargos include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

In some embodiments, the cargo is a polynucleotide. In some embodiments, the polynucleotide is an RNA or a DNA molecule.

In some embodiments, the polynucleotide is introduced into the target cells of a tissue or an organ and is capable of being expressed under appropriate conditions, or otherwise conferring a beneficial property to the cells. The polynucleotide is thus selected based upon a desired therapeutic outcome. For instance, the polynucleotide encodes for to a polypeptide that confers a beneficial property to the cells or a desired therapeutic outcome. Examples of polynucleotides of interest include but are not limited to those encoding for a polypeptide selected from the group consisting of protective polypeptides (e.g., neuroprotective polypeptides such as GDNF, CNTF, NT4, NGF, and NTN); anti-angiogenic polypeptides (e.g., a soluble vascular endothelial growth factor (VEGF) receptor; a VEGF-binding antibody; a VEGF-binding antibody fragment (e.g., a single chain anti-VEGF antibody); and anti-apoptotic polypeptides (e.g., Bcl-2, Bcl-X1); and the like.

In some embodiments, the polynucleotide encodes for an antigen. As used herein, the term “antigen” has its general meaning in the art and generally refers to a substance or fragment thereof that is recognized and selectively bound by an antibody or by a T cell antigen receptor, resulting in induction of an immune response. Antigens according to the invention are typically, although not exclusively, peptides and proteins. An antigen in the context of the invention can comprise any subunit, fragment, or epitope of any proteinaceous molecule, including a protein or peptide of viral, bacterial, parasitic, fungal, protozoan, prion, cellular, or extracellular origin, which ideally provokes an immune response in mammal, preferably leading to protective immunity. In some embodiments, the antigen is a tumor antigen. In particular, the antigen can be a peptide isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae (e.g., Norovirus (also known as “Norwalk-like virus”)), Capillovirus, Carlavirus, Caulimovirus, Circoviridae, Closterovirus, Comoviridae, Coronaviridae (e.g., Coronavirus, such as severe acute respiratory syndrome (SARS) virus, or SARS-COV-2), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae (e.g., Hepatitis B virus or Hepatitis C virus), Herpesviridae (e.g., Human herpesvirus (HSV) 1, 2, 3, 4, 5, and 6, Cytomegalovirus, and Epstein-Barr Virus (EBV)), Hypoviridae, Iridoviridae, Leviviridae, Lipothrixviridae, Microviridae, Orthomyxoviridae (e.g., Influenzavirus A and B), Papovaviridae, Papillomaviridae (e.g., human papillomavirus (HPV)), Paramyxoviridae (e.g., measles, mumps, and human respiratory syncytial virus (RSV)), Parvoviridae, Picornaviridae (e.g., poliovirus, rhinovirus, hepatovirus, and aphthovirus (e.g., foot and mouth disease virus)), Poxviridae (e.g., vaccinia virus), Reoviridae (e.g., rotavirus), Retroviridae (e.g., lentivirus, such as human immunodeficiency virus (HIV) 1 and HIV 2), Rhabdoviridae, and Totiviridae.

In some embodiments, the polynucleotide of the present invention is an RNA molecule, in particular a messenger RNA (mRNA). In some embodiments, the EV encapsuled one or more RNA molecules capable of inducing: i) transfer of one or more endogenous or exogenous coding sequences of interest of the target cell, ii) transfer of one or more non-coding RNAs such as RNAs capable of inducing an effect on genetic expression, for example by means of shRNA, miRNA, sgRNA, LncRNA or circRNA, iii) transfer of cellular RNAs, of the messenger RNA type or others (miRNA etc.), subgenomic replicons of RNA viruses (HCV, etc.) or of complete genomes of RNA viruses, iv) simultaneous expression of endogenous or exogenous coding or non-coding sequences of the target cell, or vi) participation in modification of the genome of the target cell by genome engineering systems, for example the CRISPR system.

In some embodiments, the polynucleotide is an antisense or siRNA sequence that acts to reduce expression of a targeted sequence. Antisense or siRNA nucleic acids are designed to specifically bind to RNA, resulting in the formation of RNA-DNA or RNA-RNA hybrids, with an arrest of DNA replication, reverse transcription or messenger RNA translation. Gene expression is reduced through various mechanisms. Antisense nucleic acids based on a selected nucleic acid sequence can interfere with expression of the corresponding gene. Antisense oligodeoxynucleotides (ODN), include synthetic ODN having chemical modifications from native nucleic acids, or nucleic acid constructs that express such anti-sense molecules as RNA. One or a combination of antisense molecules may be administered, where a combination may comprise multiple different sequences. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

Also of interest are RNAi agents. RNAi agents are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides.

In some embodiments, the cargo is a polynucleotide that encodes for an endonuclease, a base-editing enzyme, an epigenome editor or a prime editor as described herein after.

In some embodiments, the cargo is a polypeptide. Polypeptides of interest include biologically active proteins, e.g. transcription factors, proteins involved in signaling pathways, cytokines, chemokines, toxins, and the like. Such polypeptides may include proteins not found in the target cell, proteins from different species or cloned versions of proteins found in the target cell.

Preferred target proteins of the invention will be proteins with the same status as that found in the target cell expressed in such a way that post-translational modification is the same as that found in the target cell. Such modification includes glycosylation or lipid modification addition of coenzyme groups or formation of quaternary structure. Most preferred will be wild type proteins corresponding to proteins found in mutated form or absent in the target cell. In some embodiment, the polypeptide is a membrane protein or a non-membrane protein. Non-limiting examples of membrane proteins include ion channels, receptor tyrosine kinases such as the PDGF-receptor and the SCF-R receptor (Stem Cell Factor Receptor, or c-kit, or CD117), G-protein linked receptors such as adrenoreceptors. Non-limiting examples of non-membrane proteins include cytosolic proteins such as actin, Ras, ERK1/2 and nuclear proteins such as steroid receptors, histone proteins, or transcriptional factors.

In some embodiments, the cargo is an endonuclease that provides for site-specific knock-down of gene function. For example, where a dominant allele encodes a defective copy of a gene that, when wild-type, is structural protein and/or provides for normal function, a site-specific endonuclease can be targeted to the defective allele and knock out the defective allele. In addition to knocking out a defective allele, a site-specific nuclease can also be used to stimulate homologous recombination with a donor DNA that encodes a functional copy of the protein encoded by the defective allele. Thus, e.g., the method of the invention can be used to deliver both a site-specific endonuclease that knocks out a defective allele, and can be used to deliver a functional copy of the defective allele, resulting in repair of the defective allele, thereby providing for production of a functional protein.

In some embodiments, the DNA targeting endonuclease is a Transcription Activator-Like Effector Nuclease (TALEN). TALENs are produced artificially by fusing a TAL effector (“TALE”) DNA binding domain, e.g., one or more TALEs, e.g., 1, 2, 3, 4, 5, 6, 7, 8, 9 or 10 TALEs to a DNA-modifying domain, e.g., a FokI nuclease domain. Transcription activator-like effects (TALEs) can be engineered to bind any desired DNA sequence (Zhang (2011), Nature Biotech. 29:149-153). By combining an engineered TALE with a DNA cleavage domain, a restriction enzyme can be produced which is specific to any desired DNA sequence. These can then be introduced into a cell, wherein they can be used for genome editing (Boch (2011) Nature Biotech. 29:135-6; and Boch et al. (2009) Science 326:1509-12; Moscou et al. (2009) Science 326:3501). TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence (Zhang (2011), Nature Biotech. 29:149-153). To produce a TALEN, a TALE protein is fused to a nuclease (N), e.g., a wild-type or mutated FokI endonuclease. Several mutations to FokI have been made for its use in TALENs; these, for example, improve cleavage specificity or activity (Cermak et al. (2011) Nucl. Acids Res. 39: e82; Miller et al. (2011) Nature Biotech. 29:143-8; Hockemeyer et al. (2011) Nature Biotech. 29:731-734; Wood et al. (2011) Science 333:307; Doyon et al. (2010) Nature Methods 8:74-79; Szczepek et al. (2007) Nature Biotech. 25:786-793; and Guo et al. (2010) J. Mol. Biol. 200:96). The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity (Miller et al. (2011) Nature Biotech. 29:143-8). TALEN can be used inside a cell to produce a double-strand break in a target nucleic acid, e.g., a site within a gene. A mutation can be introduced at the break site if the repair mechanisms improperly repair the break via non-homologous end joining (Huertas, P., Nat. Struct. Mol. Biol. (2010) 17:11-16). For example, improper repair may introduce a frame shift mutation. Alternatively, foreign DNA can be introduced into the cell along with the TALEN; depending on the sequences of the foreign DNA and chromosomal sequence, this process can be used to modify a target gene via the homologous direct repair pathway, e.g., correct a defect in the target gene, thus causing expression of a repaired target gene, or e.g., introduce such a defect into a wt gene, thus decreasing expression of a target gene.

In some embodiments, the DNA targeting endonuclease is a Zinc-Finger Nuclease (ZFN). Like a TALEN, a ZFN comprises a DNA-modifying domain, e.g., a nuclease domain, e.g., a FokI nuclease domain (or derivative thereof) fused to a DNA-binding domain. In the case of a ZFN, the DNA-binding domain comprises one or more zinc fingers, e.g., 1, 2, 3, 4, 5, 6, 7, 8, 9 or 10 zinc fingers (Carroll et al. (2011) Genetics Society of America 188:773-782; and Kim et al. (1996) Proc. Natl. Acad. Sci. USA 93:1156-1160). A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art (Sera (2002), Biochemistry, 41:7074-7081; Liu (2008) Bioinformatics, 24:1850-1857). A ZFN using a FokI nuclease domain or other dimeric nuclease domain functions as a dimer. Thus, a pair of ZFNs are required to target non-palindromic DNA sites. The two individual ZFNs must bind opposite strands of the DNA with their nucleases properly spaced apart (Bitinaite et al. (1998) Proc. Natl. Acad. Sci. USA 95:10570-5). Also like a TALEN, a ZFN can create a DSB in the DNA, which can create a frame-shift mutation if improperly repaired, e.g., via non-homologous end joining, leading to a decrease in the expression of a target gene in a cell.

In some embodiments, the DNA targeting endonuclease is a CRISPR-associated endonuclease. In bacteria the CRISPR/Cas loci encode RNA-guided adaptive immune systems against mobile genetic elements (viruses, transposable elements and conjugative plasmids). Three types (I-VI) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activated small RNA (tracrRNA) that serves as a guide for ribonuclease Ill-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd or the 4th nucleotide from PAM). The crRNA and tracrRNA can be expressed separately or engineered into an artificial fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or H1-promoted RNA expression vector.

In some embodiments, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Alternatively, the wild type Streptococcus pyogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as pX330, pX260 or pMJ920 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of pX330, pX260 or pMJ920 (Addgene, Cambridge, MA).

In some embodiments, the cargo is a base-editing enzyme. As used herein, the term “base-editing enzyme” refers to fusion protein comprising a defective CRISPR/Cas nuclease linked to a deaminase polypeptide. The term is also known as “base-editor”. As used herein, the term “deaminase” refers to an enzyme that catalyses a deamination reaction. The term “deamination”, as used herein, refers to the removal of an amine group from one molecule. In some embodiments, the deaminase is a cytidine deaminase, catalysing the hydrolytic deamination of cytidine or deoxycytidine to uracil or deoxyuracil, respectively. In some embodiments, the deaminase is an adenosine deaminase, catalysing the hydrolytic deamination of adenosine to inosine, which is treated like guanosine by the cell, creating an A to G (or T to C) change. Two classes of base-editing enzymes—cytosine base-editing enzymes (CBEs) and adenine base-editing enzymes (ABEs)—can be used to generate single base pair edits without double stranded breaks. Typically, cytosine base-editing enzymes are created by fusing the defective CRISPR/Cas nuclease to a deaminase.

In some embodiments, the cargo is a prime editor that consists of a fusion protein wherein a catalytically impaired Cas9 endonuclease is fused to an engineered reverse transcriptase enzyme. By complexing a prime editing guide RNA (pegRNA), the prime editor is capable of identifying the target site and providing the new genetic information to replace the target DNA nucleotides. It mediates targeted insertions, deletions, and base-to-base conversions without the need for double strand breaks (DSBs) or donor DNA templates (Anzalone, Andrew V.; Randolph, Peyton B.; Davis, Jessie R.; Sousa, Alexander A.; Koblan, Luke W.; Levy, Jonathan M.; Chen, Peter J.; Wilson, Christopher; Newby, Gregory A.; Raguram, Aditya; Liu, David R. (21 Oct. 2019). “Search-and-replace genome editing without double-strand breaks or donor DNA”. Nature. 576 (7785): 149 157.).

In some embodiments, the EV is loaded with i) a polypeptide (or a polynucleotide encoding thereof) selected from the group consisting of CRISPR-associated endonucleases, base editing enzymes, epigenome editing factors and primer editors and ii) one or more guide RNA molecules.

As used herein, the term “guide RNA molecule” generally refers to an RNA molecule (or a group of RNA molecules collectively) that can bind to a Cas9 protein and target the Cas9 protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is sometimes referred to as the CRISPR RNA (crRNA), while the molecule comprising the protein-binding segment is referred to as the trans-activating RNA (tracrRNA).

In some embodiments, the cargo is a toxin.

As used herein, the term “toxin” refers to a molecule or moiety that is generally lethal to a cell. In some embodiments, the toxin a bacterial toxin or a fragment thereof. As used herein, the term “bacterial toxin” refers to a polypeptide produced by a pathogenic bacteria, and involved in said pathogenic activity. It may be a factor which is directly responsible for the toxicity of the bacterium, or it may participate in that toxicity. As used herein the term “toxin fragment” refers to any portion of a toxin, which has retained the toxicity activity. In particular, bacterial toxins have been described as often presenting different distinct functional domains, in particular a domain involved in toxic activity (catalytic site) distinct from other domains involved in site recognition or in interactions with partners. Most bacterial toxins, such as diphtheria toxin, Pseudomonas exotoxin, and Clostridium perfringens enterotoxin, include a receptor-binding moiety that targets the toxin to a particular cell-surface receptor, and a moiety that is responsible for the toxicity of the toxin protein. For instance, Clostridium perfringens enterotoxin binds to claudin-3 and claudin-4 on the cell surface. Clostridium perfringens enterotoxin (CPE) is a protein of 319 amino acid residues. A peptide consisting of residues 290-319 of Clostridium perfringens enterotoxin binds to claudin-3 and claudin-4 but is not toxic (Hanna, P. C., et al., 1991, J. Biol. Chem. 266:11037-43). Approximately residues 45-116 of CPE are responsible for cytolysis of cells through forming large complexes in the cell membrane (Kokai-Kun, J. F. et al., 1996, Infect. Immun. 64:1020-25; Kokai-Kun, J. F. et al., 1997, Clin. Infect. Dis. 25 (Suppl. 2): S165-5167; Kokai-Kun, J. F. et al., Infect. Immun. 65:1014-1022; Kokai-Kun, J. F. et al., 1999, Infect. Immun. 67:5634-5641; Hanna, P. C., et al., 1991, J. Biol. Chem. 266:11037-43). Deletion of just residues 315-319 is enough to abolish binding to the receptors (Kokai-Kun, J. F. et al., 1999, Infect. Immun. 67:5634-5641). Thus, in some embodiments, the toxin is a fragment of CPE containing residues 45-116 of CPE, but lacking residues 315-319 of CPE. In some embodiments, the toxin is diphtheria toxin or a toxic fragment thereof. Diphtheria toxin is a protein of 535 amino acid residues (SEQ ID NO: 4). It contains three domains: i) residues 1-193 are the catalytic domain, having the ADP-ribosyl transferase activity that is responsible for inactivating elongation factor-2 in cells to kill them (Choe, S. et al., 1992, Nature 357:216-222), ii) residues 203-378 are responsible for translocation of the toxin across the cell membrane, and ii) residues 386-535 are responsible for binding to the receptor. Thus, in a particular embodiment, the toxin of the present invention comprises amino acid sequence as set forth in SEQ ID NO: 4.

SEQ ID NO: 4 > Sequence of DT toxin
MDDVVDSSKSFVMENFSSYHGTKPGYVDSIQKGIQKPKSGTQGNYDDDWK
GFYSTDNKYDAAGYSVDNENPLSGKAGGVVKVTYPGLTKVLALKVDNAET
IKKELGLSLTEPLMEQVGTEEFIKRFGDGASRVVLSLPFAEGSSSVEYIN
NWEQAKALSVELEINFETRGKRGQDAMYEYMAQACAGNRVRRTSYPYDVP
DYA

Other cargos of interest include detectable markers, e.g. luciferase, luciferin, green fluorescent proteins, fluorochromes, e.g. FITC, etc., and the like. Detectable markers may also include imaging entities, e.g. metallic nanoparticles such as gold, platinum, silver, etc., which may be provided as nanoparticles, usually nanoparticles of less than 10 nm, less than about 5 nm, etc.

### Loading System:

In some embodiments, the EV of the present invention comprises a structural polypeptide that is capable of forming a dimer with the cargo polypeptide.

As used herein, the term “structural polypeptide” is a protein that is naturally incorporated in the membrane of the EV and that contributes to the overall structure of said EV.

In some embodiments, the structural polypeptide is selected among transmembrane proteins. As sued herein, the term “transmembrane protein” has its general meaning in the art and refers to a membrane protein that spans the lipid bilayer of the membrane. In some embodiments, the transmembrane protein is a tetraspanin. As used herein, the term “tetraspanin” has its general meaning in the art and refers to to a superfamily of small, four transmembrane domain proteins that are involved in very diverse physiological processes. Members of tetraspanin include but are not restricted to CD9, CD37, CD53, CD63, CD81 and CD82. In some embodiments, the tetraspanin is CD63.

The means by which the structural polypeptide and the cargo polypeptide form a dimer is not particularly limited. In some embodiments, the structural polypeptide and the cargo polypeptide (e.g. the toxin) are fused either directly or via a linker to respective domains that are capable of dimerization in presence of a compound. For instance, it is possible to use a system in which a FK506-binding protein (“FKBP domain”) and a FKBP-rapamycin-associated protein 1, FRAP1 fragment (“FRB domain”) form a heterodimer in the presence of rapamycin. Thus, in some embodiments, the structural polypeptide is fused to the FKBP domain and the cargo polypeptide (e.g. the toxin) is fused to the FRB domain (or vice-versa), it is possible to dimerize the FKBP domain and the FRB domain in presence of rapamycin during the production of the EVs of the present invention. In some embodiments, the FKBP domain consists of the amino acid sequence as set forth in SEQ ID NO: 5 and the FRB domain consists of the amino acid sequence as set forth in SEQ ID NO: 6.

SEQ ID NO: 5 > FKBP2 domain
GVQVETISPGDGRTFPKRGQTCVVHYTGMLEDGKKFDSSRDRNKPFKFM
LGKQEVIRGWEEGVAQMSVGQRAKLTISPDYAYGATGHPGIIPPHATLV
FDVELLKLETRGVQVETISPGDGRTFPKRGQTCVVHYTGMLEDGKKFDS
SRDRNKPFKFMLGKQEVIRGWEEGVAQMSVGQRAKLTISPDYAYGATGH
PGIIPPHATLVFDVELLKLE
SEQ ID NO: 6 > FRB domain
ILWHEMWHEGLEEASRLYFGERNVKGMFEVLEPLHAMMERGPQTLKETS
FNQAYGRDLMEAQEWCRKYMKSGNVKDLLQAWDLYYHVFRRISK

Alternatively, it is possible to use a system in which GAI (Gibberellin insensitive) and GID1 (Gibberellin insensitive dwarf1) form a heterodimer in the presence of gibberellin or GA3-AM (for example, see Miyamoto T., et al., Rapid and Orthogonal Logic Gating with a Gibberellin-induced Dimerization System, Nat Chem Biol., 8 (5), 465-470, 2012), a system in which PyL (PYR1-like, consisting of the 33rd to 209th amino acids) and ABI1 (consisting of the 126th to 423rd amino acids) form a heterodimer in the presence of S-(+)-abscisic acid (ABA) (for example, see, Liang F. S., et al., Engineering the ABA plant stress pathway for regulation of induced proximity, Sci Signal., 4 (164), rs2, 2011), and the like.

In some embodiments, the EV of the present invention comprises a loading system wherein the tetraspanin CD63 is fused to the FKBP2 domain. In some embodiments, the EV of the present invention comprises a loading system that consists of the amino acid sequence as set forth in SEQ ID NO: 7. In some embodiments, the cargo polypeptide (e.g. the toxin) is fused to the FRB domain and thus can dimerize with the CD63-FKBP2 fusion protein in presence of rapamycin; allowing the loading of the cargo polypeptide into the EV.

SEQ ID NO: 7 > FKBP2-FRP-CD63 amino acid sequence
MASRGVQVETISPGDGRTFPKRGQTCVVHYTGMLEDGKKFDSSRDRNKP
FKFMLGKQEVIRGWEEGVAQMSVGQRAKLTISPDYAYGATGHPGIIPPH
ATLVFDVELLKLETRGVQVETISPGDGRTFPKRGQTCVVHYTGMLEDGK
KFDSSRDRNKPFKFMLGKQEVIRGWEEGVAQMSVGQRAKLTISPDYAYG
ATGHPGIIPPHATLVFDVELLKLETRMASSEDVIKEFMRFKVRMEGSVN
GHEFEIEGEGEGRPYEGTQTAKLKVTKGGPLPFAWDILSPQFQYGSKAY
VKHPADIPDYLKLSFPEGFKWERVMNFEDGGVVTVTQDSSLQDGEFIYK
VKLRGTNFPSDGPVMQKKTMGWEASTERMYPEDGALKGEIKMRLKLKDG
GHYDAEVKTTYMAKKPVQLPGAYKTDIKLDITSHNEDYTIVEQYERAEG
RHSTGALYKSGLRSRAMAVEGGMKCVKELLYVLLLAFCACAVGLIAVGV
GAQLVLSQTIIQGATPGSLLPVVIIAVGVFLFLVAFVGCCGACKENYCL
MITFAIFLSLIMVEVAAAIAGYVFRDKVMSEFNNNFRQQMENYPKNNHT
ASILDRMQADFKCCGAANYTDWEKIPSMSKNRVPDSCCINVTVGCGINF
NEKAIHKEGCVEKIGGWLRKNVLVVAAAALGIAFVEVLGIVFACCLVKS
IRSGYEVM

### Targeting Moiety:

According to the present invention, the targeting moiety is a polypeptide having a binding domain. The term “binding domain” as used herein refers to the one or more regions of a polypeptide that mediate specific binding with a target molecule (e.g. an antigen, ligand, receptor, substrate or inhibitor). Exemplary binding domains include an antibody variable domain, a receptor binding domain of a ligand, a ligand binding domain of a receptor or an enzymatic domain. The term “ligand binding domain” as used herein refers to any native receptor (e.g., cell surface receptor) or any region or derivative thereof retaining at least a qualitative ligand binding ability of a corresponding native receptor. The term “receptor binding domain” as used herein refers to any native ligand or any region or derivative thereof retaining at least a qualitative receptor binding ability of a corresponding native ligand. In some embodiments, the polypeptide comprises at least 1, 2, 3, 4, or 5 binding sites. The polypeptide may be either monomers or multimers. For example, in some embodiments, the polypeptide is a dimer. In some embodiments, the dimer is a homodimer, comprising two identical monomeric subunits. In some embodiments, the dimer is a heterodimer, comprising two non-identical monomeric subunits. The subunits of the dimer may comprise one or more polypeptide chains. For example, in some embodiments, the dimer comprises at least two polypeptide chains. In some embodiments, the dimer comprises two polypeptide chains. In some embodiments, the dimer comprises four polypeptide chains (e.g., as in the case of antibody molecules).

In some embodiments, the targeting moiety is a ligand.

In some embodiments, the targeting moiety is an antibody or an antibody-fragment such as an scFv or VHH or other functional fragment including an immunoglobulin devoid of light chains, Fab, Fab′, F(ab*)2, Fv, antibody fragment, diabody, scAB, single-domain heavy chain antibody, single-domain light chain antibody, Fd, CDR regions, or any portion or peptide sequence of the antibody that is capable of binding antigen or epitope. Thus, in some embodiments, the polypeptide having a binding domain is a light immunoglobulin chain. In some embodiments, the polypeptide having a binding domain is a heavy immunoglobulin chain. In some embodiments, the polypeptide having a binding domain is a heavy single heavy chain variable domain of antibodies of the type that can be found in Camelid mammals which are naturally devoid of light chains. Such single domain antibody is also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21 (11): 484-490; and WO 06/030220, WO 06/003388.

The techniques for preparing and using various antibody-based constructs and fragments are well known in the art (see e.g. Kohler and Milstein, Nature, 256:495, 1975).

In some embodiments, the antibody is a monoclonal antibody.

In some embodiments, the targeting moiety has binding affinity to a cell surface molecule of a target cell. In some embodiments, the cell surface molecule is a receptor. In some embodiments, the cell surface molecule is a transmembrane protein. In some embodiments, the target moiety is specific for target protein antigens, carbohydrate antigens, or glycosylated proteins. For example, the antibody can target glycosylation groups of antigens that are preferentially produced by transformed (neoplastic or cancerous) cells, infected cells, and the like (cells associated with other immune system-related disorders).

A partial list of suitable mammalian cells that can be targeted by the targeting moiety of the present invention includes but are not limited to blood cells, myoblasts, bone marrow cells, peripheral blood cells, umbilical cord blood cells, cardiomyocytes (and precursors thereof), chondrocytes (cartilage cells), dendritic cells, fetal neural tissue, fibroblasts, hepatocytes (liver cells), islet cells of pancreas, keratinocytes (skin cells) and stem cells.

In some embodiments, the targeting moiety is particularly suitable for targeting a population of malignant cells. Thus, in some embodiments, the targeting moiety is specific for a cancer antigen. Known cancer antigens include, without limitation, c-erbB-2 (erbB-2 is also known as c-neu or HER-2), which is particularly associated with breast, ovarian, and colon tumor cells, as well as neuroblastoma, lung cancer, thyroid cancer, pancreatic cancer, prostate cancer, renal cancer and cancers of the digestive tract. Another class of cancer antigens is oncofetal proteins of nonenzymatic function. These antigens are found in a variety of neoplasms, and are often referred to as “tumor-associated antigens.” Carcinoembryonic antigen (CEA), and a-fetoprotein (AFP) are two examples of such cancer antigens. AFP levels rise in patients with hepatocellular carcinoma: 69% of patients with liver cancer express high levels of AFP in their serum. CEA is a serum glycoprotein of 200 kDa found in adenocarcinoma of colon, as well as cancers of the lung and genitourinary tract. Yet another class of cancer antigens is those antigens unique to a particular tumor, referred to sometimes as “tumor specific antigens” such as heat shock proteins (e.g., hsp70 or hsp90 proteins) from a particular type of tumor. Other targets include the MICA/B ligands of NKG2D. These molecules are expressed on many types of tumors, but not normally on healthy cells. Additional specific examples of cancer antigens include epithelial cell adhesion molecule (Ep-CAM/TACSTD1), mesothelin, tumor-associated glycoprotein 72 (TAG-72), gp100, Melan-A, MART-1, KDR, RCAS1, MDA7, cancer-associated viral vaccines (e.g., human papillomavirus antigens), prostate specific antigen (PSA, PSMA), RAGE (renal antigen), CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), cancer-associated ganglioside antigens, tyrosinase, gp75, C-myc, Mart1, MelanA, MUM-1, MUM-2, MUM-3, HLA-B7, Ep-CAM, tumor-derived heat shock proteins, and the like (see also, e.g., Acres et al., Curr Opin Mol Ther 2004 Feb. 6:40-7; Taylor-Papadimitriou et al., Biochim Biophys Acta. 1999 Oct. 8; 1455 (2-3): 301-13; Emens et al., Cancer Biol Ther. 2003 July-August; 2 (4 Suppl 1): S161-8; and Ohshima et al., Int J Cancer. 2001 Jul. 1; 93 (1): 91-6). Other exemplary cancer antigen targets include CA 195 tumor-associated antigen-like antigen (see, e.g., U.S. Pat. No. 5,324,822) and female urine squamous cell carcinoma-like antigens (see, e.g., U.S. Pat. No. 5,306,811), and the breast cell cancer antigens described in U.S. Pat. No. 4,960,716.

In some embodiments, the targeting moiety has binding affinity for a CD (cluster of differentiation) molecule selected from the group consisting of CD1a, CD1b, CD1c, CD1d, CD1e, CD2, CD3delta, CD3epsilon, CD3gamma, CD4, CD5, CD6, CD7, CD8alpha, CD8beta, CD9, CD10, CD11a, CD11b, CD11c, CDw12, CD13, CD14, CD15u, CD16a, CD16b, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD44R, CD45, CD46, CD47R, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD65s, CD66a, CD66b, CD66c, CD66d, CD66e, CD66f, CD68, CD69, CD70, CD71, CD72, CD73, CD74, CD75, CD75s, CD77, CD79a, CD79b, CD80, CD81, CD82, CD83, CD84, CD85, CD86, CD87, CD88, CD89, CD90, CD91, CD92, CDw93, CD94, CD95, CD96, CD97, CD98, CD99, CD100, CD101, CD102, CD103, CD104, CD105, CD106, CD107a, CD107b, CD108, CD109, CD110, CD111, CD112, CDw113, CD114, CD115, CD116, CD117, CD118, CDw119, CD120a, CD120b, CD121a, CDw121b, CD122, CD123, CD124, CDw125, CD126, CD127, CDw128a, CDw128b, CD129, CD130, CD131, CD132, CD133, CD134, CD135, CDw136, CDw137, CD138, CD139, CD140a, CD140b, CD141, CD142, CD143, CD144, CDw145, CD146, CD147, CD148, CDw149, CD150, CD151, CD152, CD153, CD154, CD155, CD156a, CD156b, CDw156C, CD157, CD158, CD159a, CD159c, CD160, CD161, CD162, CD162R, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CDw186, CD191, CD192, CD193, CD195, CD196, CD197, CDw198, CDw199, CDw197, CD200, CD201, CD202b, CD203c, CD204, CD205, CD206, CD207, CD208, CD209, CDw210, CD212, CD213a1, CD213a2, CDw217, CDw218a, CDw218b, CD220, CD221, CD222, CD223, CD224, CD225, CD226, CD227, CD228, CD229, CD230, CD231, CD232, CD233, CD234, CD235a, CD235b, CD235ab, CD236, CD236R, CD238, CD239, CD240CE, CD240D, CD240DCE, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD271, CD272, CD273, CD274, CD275, CD276, CD277, CD278, CD279, CD280, CD281, CD282, CD283, CD284, CD289, CD292, CDw293, CD294, CD295, CD296, CD297, CD298, CD299, CD300a, CD300c, CD300e, CD301, CD302, CD303, CD304, CD305, CD306, CD307, CD309, CD312, CD314, CD315, CD316, CD317, CD318, CD319, CD320, CD321, CD322, CD324, CDw325, CD326, CDw327, CDw328, CDw329, CD331, CD332, CD333, CD334, CD335, CD336, CD337, CDw338, and CD339.

### Methods for Producing the EVs of the Present Invention:

According to the present invention, the EV of the present invention is prepared from a donor that has been genetically engineered to express the components of the EV, namely the ERV syncytin, the cargo(s) of interest, and optionally the loading system and the targeting moiety as well. Typically, the donor cell is transduced in order to express one or more polynucleotide that encodes for the different components of the EV. It is contemplated that the polynucleotide construct can be introduced into the donor cells as naked DNA or in a suitable vector. Naked DNA generally refers to the DNA contained in a plasmid expression vector in proper orientation for expression. Physical methods for introducing a polynucleotide construct into a donor cell include calcium phosphate precipitation, lipofection, particle bombardment, microinjection, electroporation, and the like. Other means can be used including colloidal dispersion systems, such as macromolecule complexes, nanocapsules, microspheres, beads, and lipid-based systems including oil-in-water emulsions, micelles, mixed micelles, and liposomes. In some embodiments, the polynucleotide construct is introduced into the donor cell by a viral vector that is an adeno-associated virus (AAV), a retrovirus, lentivirus, bovine papilloma virus, an adenovirus vector, a vaccinia virus, a polyoma virus, or an infective virus. In some embodiments, the vector is a retroviral. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, the polynucleotide is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. Typically, the vector of the present invention include “control sequences′”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”. In some embodiments, the polynucleotide is encoded by a nucleic acid molecule whose sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148. In order to confirm the presence of the polynucleotide in the donor cell, a variety of assays may be performed. Such assays include, for example, “molecular biological” assays well known such as Southern and Northern blotting, RT-PCR and quantitative PCR; or “biochemical” assays, such as detecting the presence or absence of a particular peptide.

Donor cells include without limitation epithelial cells, circulating immune cells, hematopoietic cells, bone marrow cells, circulating vascular progenitor cells, cardiac cells, chondrocytes, bone cells, beta cells, hepatocytes, and neurons . . . . Moreover the donor cells includs pluripotent stem cells. As intended herein, the expression “pluripotent stem cells” relates to division-competent cells which are liable to differentiate in one or more cell types. Preferably, the pluripotent stem cells are not differentiated. Pluripotent stem cells encompass stem cells, in particular adult stem cells (e.g. mesenchymal stem cells (MSC)) and embryonic stem cells. The term also encompasses induced pluripotent stem cells (IPS). In some embodiments, the donor cell is a mesenchymal stem cell. As used, herein, the term “mesenchymal stem cell” or “MSC” has its general meaning in the art and refers to multipotent stromal cells that can differentiate into a variety of cell types, including: osteoblasts (bone cells), chondrocytes (cartilage cells), myocytes (muscle cells) and adipocytes (fat cells) (See for example Wang, Stem Cells 2004; 22 (7); 1330-7; McElreavey; 1991 Biochem Soc Trans (1); 29s; Takechi, Placenta 1993 March/April; 14 (2); 235-45; Takechi, 1993; Kobayashi; Early Human Development; 1998; July 10; 51 (3); 223-33; Yen; Stem Cells; 2005; 23 (1) 3-9). In some embodiments, the donor cells include purified primary cells and immortalized cell lines. In some embodiments, the donor cells are cells in suspension (e.g. circulating leukocytes (PBMC)), or adherents cells (e.g. endothelial cells).

In some embodiments, the EVs of the present invention are prepared by any method well known in the art. In some embodiments, the EVs of the present invention are prepared by methods for 3D culture that are well known in the art, and include, but are not limited to standard culture in 2D flasks, hanging drop culture, culturing on matrices, culturing on microcarriers, culturing on synthetic extracellular scaffolds, culturing on chitosan membranes, culturing under magnetic levitation, suspension culture in rotating bioreactors, or culturing under non-contact inhibition conditions. See, e.g., Haycock J W. (2011). “3D cell culture: a review of current approaches and techniques.”. Methods Mol Biol. 695:1-15; Lee, J; Cuddihy M J, Kotov N A. (14 Mar. 2008). Three-dimensional cell culture matrices: state of the art. doi: 10.1089/teb.2007.0150; Pampaloni, Francesco (October 2007). “The third dimension bridges the gap between cell culture and live tissue”. Nature Reviews 8:839-845; and Souza, Glauco (14 Mar. 2010). “Three-dimensional tissue culture based on magnetic cell levitation”. Nature Nanotechnology: 291-296; the entire content of each are hereby incorporated by reference.

In some embodiments, the EVs of the present invention are prepared by the system culture described in WO2019/002608. In particular, the EVs of the present invention are prepared according to the method described in the EXAMPLE. More particularly, the method involves a fluid system comprising at least one container, a liquid medium contained by the container and producer cells, characterized in that it also comprises microcarriers suspended in the liquid medium, the majority of the producer cells being adherent to the surface of the microcarriers, and a liquid medium agitator, the agitator and the dimensions of the container being capable of controlling a turbulent flow of the liquid medium in the container. Thus, a further object of the present invention thus relates to a method of preparing a EV of the present invention comprising the steps consisting of i) causing a turbulent flow of a culture medium in a container, wherein the culture medium comprises the donor cells adhering to the surface of microcarriers, the microcarriers being in suspension in the culture medium that optionally comprises an amount of the dimerizer (e.g. rapamycin) for loading the cargo polypeptides into the EV, and then ii) collecting the produced EVs from the liquid medium. Typically, the microcarriers are microbeads. Commercially available media may be used for the growth, culture and maintenance of donor cells. Such media include but are not limited to Dulbecco's modified Eagle's medium (DMEM).

### Therapeutic Uses:

The present invention provides compositions and kits suitable for use in therapy (in vivo or ex vivo), said compositions and kits comprising an amount of the EVs of the present invention. According to the present invention, the therapeutical effects are brought by the one or more cargo(s) that is (are) loaded in the EVs of the present invention. For instance, the EVs as well as the compositions comprising them may be used for gene therapy or vaccine purposes.

Thus a further object of the present invention relates to a method of therapy in a subject in need thereof comprising administering to the subject a therapeutically amount of the EV of the present invention.

Types of diseases and disorders that can be treated by methods of the present invention include, but are not limited to infectious diseases, autoimmune diseases, inflammatory diseases, cancers, neurological diseases, cardiovascular disease, eye diseases, ear diseases, blood diseases, bone diseases, congenital diseases, metabolic diseases, musculoskeletal diseases, gastrointestinal diseases, renal and urogenital diseases, respiratory diseases, or skin diseases.

In particular the EVs of the present invention, in particular the EVs that are loaded with a toxin are particularly suitable for the treatment of cancer.

As used herein, the term “cancer” has its general meaning in the art and refers to one or more cells which are growing or have grown in an uncontrolled manner to form cancer tissue. The term includes, but is not limited to, solid tumors and blood borne tumors. The terms “cancer” and “tumor” are used interchangeably throughout the subject specification. The term “cancer” is not limited to any stage, grade, histomorphological feature, invasiveness, aggressiveness or malignancy of an affected tissue or cell aggregation. In particular stage 0 cancer, stage I cancer, stage II cancer, stage III cancer, stage IV cancer, grade I cancer, grade II cancer, grade III cancer, malignant cancer and primary carcinomas are included. As used herein, the term “solid cancer” includes, but is not limited to “carcinomas”, “adenocarcinomas” and “sarcomas”. “Sarcomas” are cancers of the connective tissue, cartilage, bone, muscle, and so on. “Carcinomas” are cancers of epithelial (lining) cells. “Adenocarcinoma” refers to carcinoma derived from cells of glandular origin.

Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

A further object of the present invention relates to a composition that comprises an amount of the EVs of the present invention (“EV composition”). Compositions as described herein encompass pharmaceutical compositions that are used for the purpose of performing a method of therapy in subject in need thereof, which includes non-human mammals and human individuals in need thereof. Compositions of the invention may be formulated for delivery to animals for veterinary purposes (e.g., livestock such as cattle, pigs, etc), and other non-human mammalian subjects, as well as to human subjects. For instance, the EVs may be formulated with a physiologically acceptable carrier for use in gene transfer and gene therapy applications. In some embodiments, the said composition further comprises one or more transduction helper compounds. The transduction helper compounds are preferably selected in a group comprising cationic polymers, as described notably by Zuris et al. (2015, Nat Biotechnol, Vol. 33 (n°1): 73-80). The transduction helper compound may be selected in a group comprising polybrene (that may be also termed hexadimethrine bromide), protamine sulfate, 12-myristate 13-acetate (also termed phorbol myristate acetate or PMA, as described by Johnston et al., 2014, Gene Ther, Vol. 21 (12): 1008-1020), vectofusin (as described by Fenard et al., 2013, Molecular Therapy Nucleic Acids, Vol. 2: e90), poloxamer P338 (as described by Anastasov et al., 2016, Lentiviral vectors and exosomes as gene and protein delivery tools, in Methods in Molecular Biology, Vol. 1448:49-61), RetroNectin® Reagent (commercialized by Clontech Laboratories Inc.), Viral Plus® transduction enhancer (commercialized by Applied Biological Materials Inc.), TransPlus® Virus Transduction Enhancer (commercialized by Clinisciences), Lentiboost® (commercialized by Sirion Biotech), or ExpressMag® Transduction System (commercialized by Sigma-Aldrich). As shown in the examples herein, the said cationic transduction helper compound may consist of polybrene. The EVs may be formulated in a conventional manner using one or more physiologically acceptable carriers or excipients. The EVs may be formulated for parenteral administration by injection, e.g., by bolus injection or continuous infusion. Formulations for injection may be presented in unit dosage form, e.g., in ampoules or in multi-dose containers, with an added preservative. The EV compositions may take such forms as suspensions, solutions or emulsions in oily or aqueous vehicles, and may contain formulation agents such as suspending, stabilizing and/or dispersing agents. Liquid preparations of the EV compositions may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts. Alternatively, the compositions may be in powder form for constitution with a suitable vehicle, e.g., sterile pyrogen-free water, before use.

The EV compositions of the invention may be administered to a subject at therapeutically effective doses to provide the therapeutic effects. In some embodiments, an amount of EV composition of the invention is administered at a dose unit that is in the range of about 0.1-5 micrograms (μg)/kilogram (kg). To this end, the EV composition of the invention may be formulated in doses in the range of about 7 mg to about 350 mg to treat to treat an average subject of 70 kg in body weight. The amount of EV composition of the invention that may be administered may be selected in a group comprising 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg or 5.0 mg/kg. The dose of EVs in a unit dosage of the composition may be selected in a group comprising 7 mg, 8 mg, 9 mg, mg, 20 mg, 25 mg, 30 mg, 35 mg, 40 mg, 45 mg, 50 mg, 55 mg, 60 mg, 65 mg, 70 mg, 75 mg, 80 mg, 85 mg 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, or 750 mg, especially for treating an average subject of 70 kg in body weight. These doses can be given once or repeatedly, such as daily, every other day, weekly, biweekly, or monthly. In some embodiments, the composition may be administered to a subject in one dose, or in two doses, or in three doses, or in four doses, or in five doses, or in six doses or more. The interval between dosages may be determined based the practitioner's determination that there is a need thereof.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. In some embodiments, the composition may be in liquid or solid (e.g. lyophilized) form.

Administration of the EVs to a human subject or an animal in need thereof can be by any means known in the art for administering virus vectors. Exemplary modes of administration include rectal, transmucosal, topical, transdermal, inhalation, parenteral (e.g., intravenous, subcutaneous, intradermal, intramuscular, and intraarticular) administration, and the like, as well as direct tissue or organ injection, alternatively, intrathecal, direct intramuscular, intraventricular, intravenous, intraperitoneal, intranasal, or intraocular injections. Injectables can be prepared in conventional forms, either as liquid solutions or suspensions, solid forms suitable for solution or suspension in liquid prior to injection, or as emulsions. Alternatively, one may administer the virus in a local rather than systemic manner, for example, in a depot or sustained-release formulation.

The invention will be further illustrated by the following figures and examples. However, these examples and figures should not be interpreted in any way as limiting the scope of the present invention.

### FIGURES

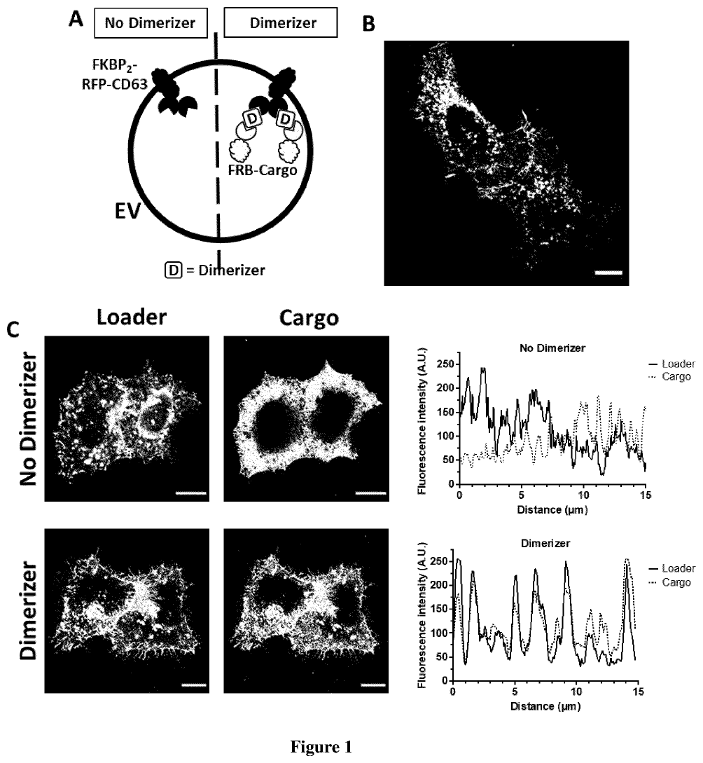

FIG. 1: EV-Cargo loading system
(A) Scheme illustrating the loading system. FKBP2-RFP-CD63, an EV membrane marker is co expressed with FRB-tagged NanoLuciferase, a luminescent reporter. Drug induced FKBP2-FRB interaction is reversible and enable EV-cargo loading. Fate the of the luminescent cargo can be follow within the extracellular media and within recipient cell through luminometry. (B) FKBP2-RFP-CD63 (Loader) was transiently expressed in HeLa WT and monitored by confocal microscopy. As expected, the loader shows an endosomal pattern. (C) Loader and FRB-NLuc cargo (which is also HA-tagged) were transiently expressed in HeLa WT cells. Transfected cells were incubated or not for 1h or not with the dimerizing drug (Dimerizer or No Dimerizer). Cells were fixed, labelled and monitored by confocal microscopy. The fluorescence intensity of both loader and cargo were plotted. These results show a significant co-localization of the two signals only in presence of the drug demonstrating the loading system ability to recruit FRB-fused proteins.

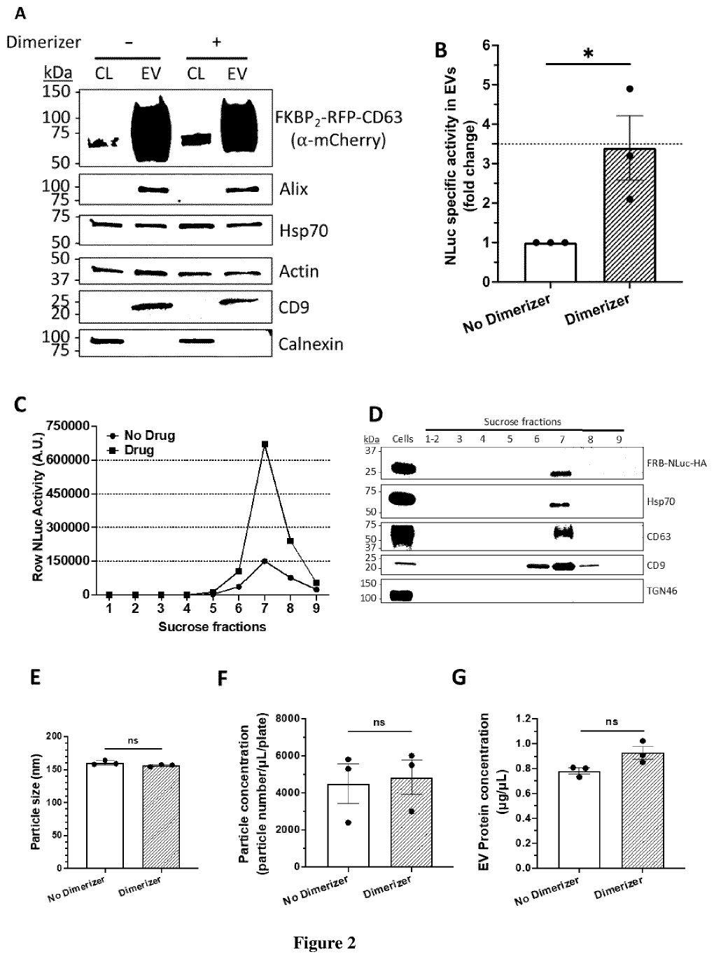

FIG. 2: drug-inducible recruitment of cargo into EVs
(A) Loader and FRB-NLuc cargo were transiently expressed in HeLa WT, and EVs were produced by these cells in the presence (Dimerizer+) or absence (Dimerizer−) of the dimerizing drug. EV were isolated by sequential centrifugation. A western blot was performed to analyze loader and cargo expression, and classic positive and negative EV markers. Each well was loaded with the same amount of protein. Dimerizing drug does not change EV composition as judged by western blot. (B) In parallel, luminescence associated within isolated EVs was measured. Graph show the luminescence activity within EV emanating from cell treated or not with the dimerizer. NLuc specific activity was normalized on the ‘No Drug’ condition, which corresponds to unspecific bulk loading of overexperessed cargo, and plotted. Each dot represents the mean of two technical duplicates. A significant 3.5-fold increase of EV NLuc specific activity is observed when donor cells are treated with the ‘Drug’. (C) A floatation assay was performed on ‘No Drug’ and ‘Drug’ EVs. The respective NLuc activity in each fraction was plotted. Both conditions show a pick of NLuc activity in the Fraction 7 which demonstrates that NLuc is associated with floating EVs. We also observe a 4-fold increase of NLuc activity of the ‘Drug’ condition in the Fraction 7 compared to the ‘No Drug’ condition confirming FIG. 2B. (D) Fractions obtained after floatation were analyzed by western blot by monitoring the cargo, and different positive and negative EV markers. Results show that the NLuc of the Fraction 7 is associated to EVs. (E-F) Particle size and concentration were measured by Nanoparticle Tracking Analysis, and (G) EV protein concentration by BCA. Each dot represents the mean of a technical duplicate. These last three parameters do not present any alteration when donor cells are treated with the drug (Dimerizer).

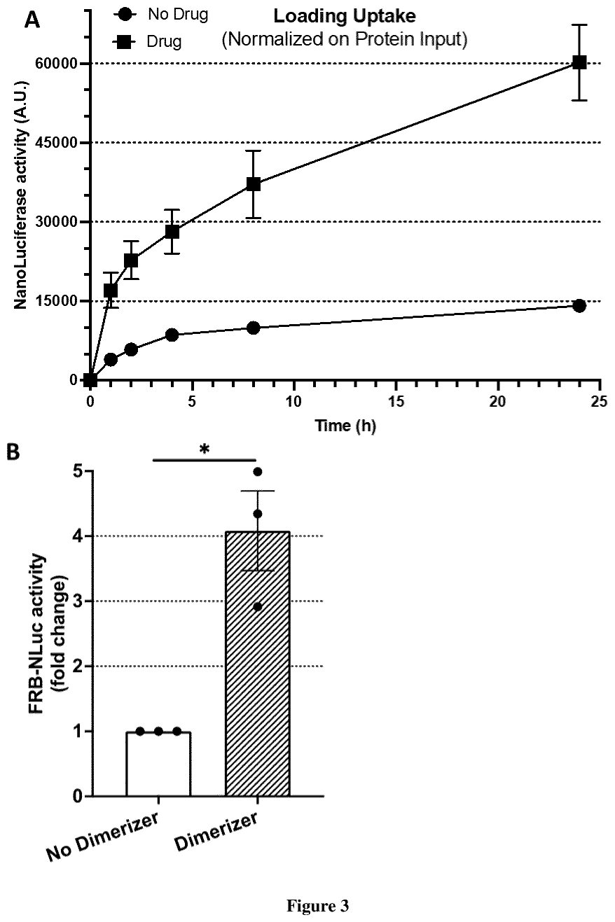

FIG. 3. EV cargo loading lead to increase uptake and delivery within receipient cells
(A) An uptake experiment was performed at different time points by incubating HeLa WT with Loading EVs produced in presence (Drug) or not (No Drug) of the dimerizing drug. Note that the dimerizing drug was washed out during EV isolation, allowing putative delivery within acceptor cell. Graph shows the luminescent activity overtime. Each point corresponds to the mean of a biological triplicate of technical duplicates, and SEM error is represented. When produced in presence of dimerizing drug, Loading EVs are capable to mediate an uptake 4-fold higher than the ‘No Drug’ condition. (B) A content delivery assay was performed by incubating during 24h HeLa WT with Loading EVs produced in presence (Dimerizer) or not (No Dimerizer) of the dimerizer drug. Briefly acceptor cells were mechanically disrupted and presence of the luminescent cargo was tested within membrane and cytosolic fractions after cell fractionation. The NLuc activity associated with membrane and cytosolic fractions of the ‘Drug’ condition were normalized on the ‘No Drug’ (No Dimerizer) condition. Results confirm a 4-fold increase of global uptake (Membrane+Cytosol).

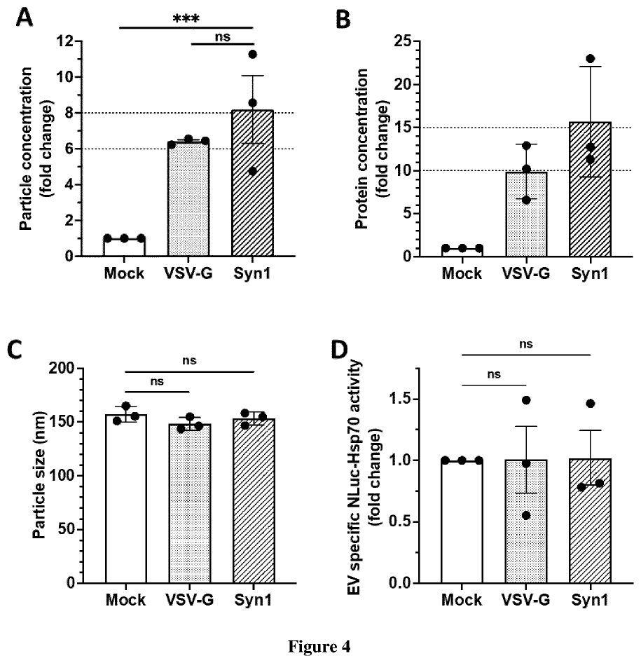

FIG. 4. Engineering virus free-fusogenic EVs
EVs from HeLa transiently expressing GFP (‘Mock condition’), VSV-G or Syncytin-1 (Syn1) were characterized according different parameters. Note that the donor cell stably express, NLuc-HSP70 a generic EV cargo. (A) Particle concentration, (B) EV protein concentration, (C) particle size and (D) EV specific NLuc activity were measured and plotted. All parameters, except particle size, were normalized on the ‘Mock’ condition. Results show an increase of particle and protein concentrations for VSV-G and Syn1 conditions. Nevertheless, EV size and specific NLuc activity are unchanged, suggesting that the EV loading capacity is not modified.

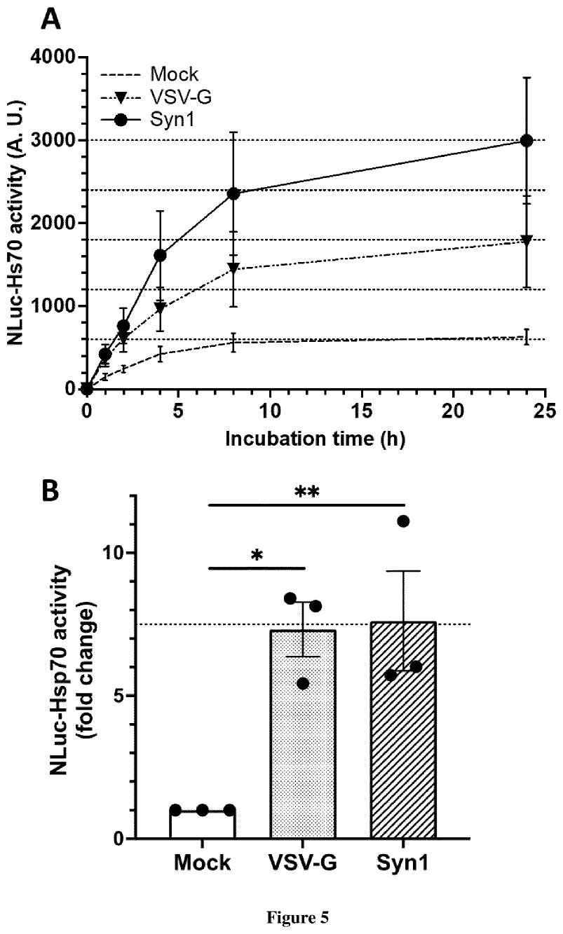

FIG. 5. SYN1 positive fusogenic Ev increase EV cargo delivery
(A) A measure of NLuc-Hs70 activity has been performed at different time points by incubating HeLa WT with EVs carrying NLuc-Hsp70 with either GFP (‘Mock’ condition), VSV-G or Syn1. Syn1-EV-mediated uptake demonstrates a significant increase compared to the ‘Mock’ condition. (B) A content delivery assay by cell fractionation was performed by incubating during 24h HeLa WT with Fusogenic EVs harboring GFP (‘Mock’ condition), VSV-G or Syn1. Results show a content delivery increased of 5-fold compared to the control for Fusogenic EVs, and a comparable EV delivery for both VSV-G and Syn1.

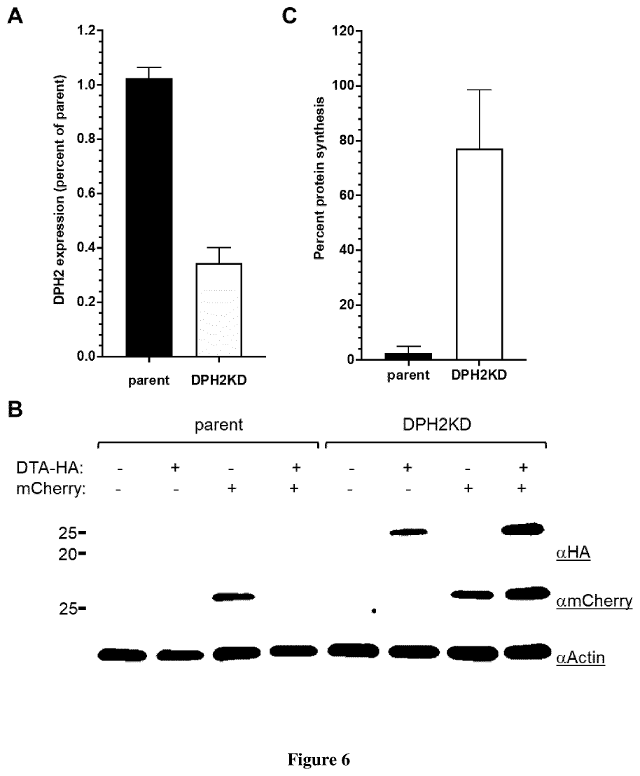

FIG. 6. DTA-resistant donor cells
(A) Parent Hela cells were infected with lentivirus-encoded shRNA targeting the DPH2 gene to generate DTA-resistant donor cells (DPH2KD). DPH2 knockdown in donor cells was confirmed by qRT-PCR. (B) A plasmid encoding DTA-HA and/or a plasmid encoding mCherry were transfected into parent or DPH2KD cells. Equal protein amounts of each sample were analyzed by western blot. While DTA-HA expression inhibits protein synthesis in parent cells (no mCherry detected), including synthesis of detectable amounts of DTA-HA itself, DPH2KD enables expression, not only of DTA-HA, but also co-expression of mCherry. This indicates that DPH2KD cells are resistant to DTA-HA-induced protein synthesis block. (C) A quantitative protein synthesis assay indicates that DPH2KD cells maintain almost 80% of de novo protein synthesis upon DTA-HA expression. In these conditions, parent cells show less than 3% of de novo protein synthesis.

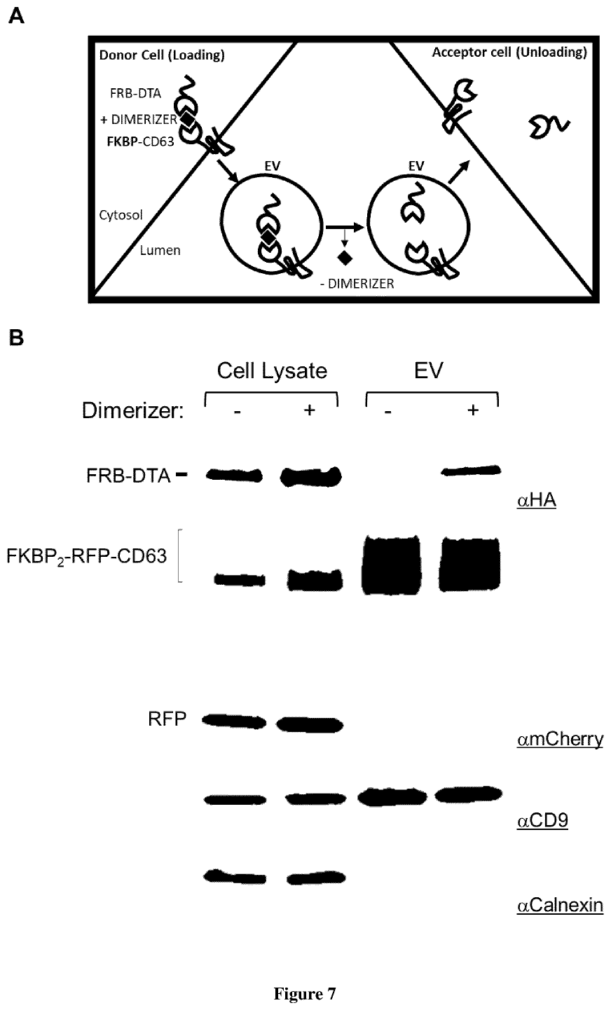

FIG. 7. Heterodimerization-dependent DTA loading into EVs.
(A) Scheme of heterodimerization-dependent DTA loading into EVs. FRB-DTA and FKBP-CD63 are co-expressed in donor cells. Upon addition of dimerizer, the two proteins bind to each other, which recruits FRB-DTA to the membrane of EVs during their biogenesis, enabling efficient FRB-DTA loading into EVs. FRB-DTA-loaded EVs may deliver their content into acceptor cell cytoplasm upon dimerizer washout. (B) Western blot analysis indicate that FRB-DTA is efficiently loaded into EVs upon addition of the dimerizer in DPH2KD donor cells. Equal amounts of protein were loaded for each sample.

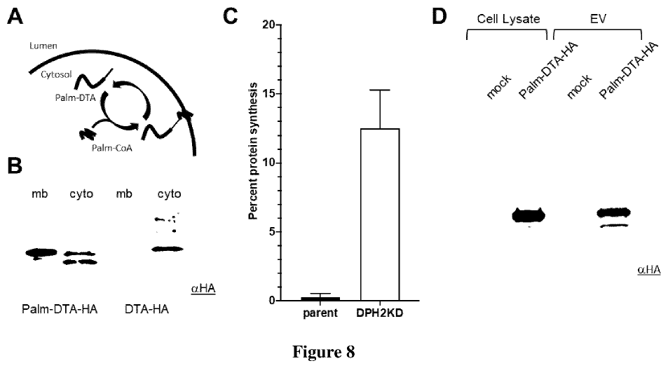

FIG. 8. Palm-DTA loading into EVs.
(A) Scheme of Palm-DTA association with membranes. (B) Cellular fractionation indicates that Palm-DTA is mostly associated with membranes, with only a minority of the protein soluble in the cytosol, contrary to DTA-HA which is only found in a soluble form. (C) A quantitative protein synthesis assay indicates that Palm-DTA is highly potent in parent cells, whereas DPH2KD cells are partially resistant to its activity. (D) Western blot analysis reveal that Palm-DTA is efficiently loaded into EVs when expressed in DPH2KD donor cells.

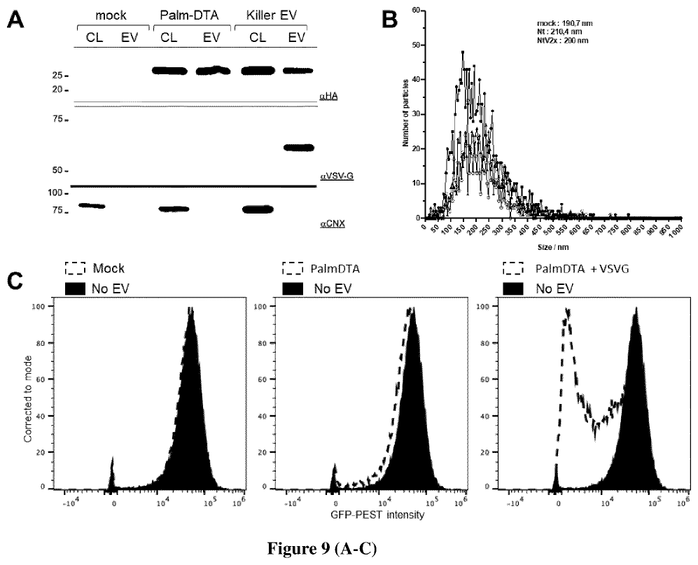

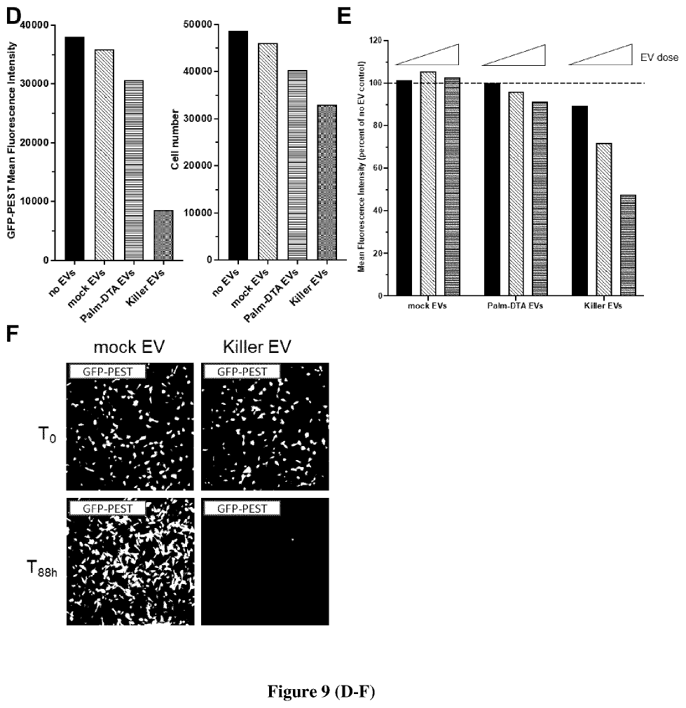

FIG. 9. Killer EVs are potent in vitro.
(A) Western blot characterization of EVs generated from DPH2KD donor cells expressing either Palm-DTA, Palm-DTA+VSV-G (Killer EVs), or none (mock). (B) Particle metrics obtained for the EVs in A. (C) The indicated EVs were incubated for 24 hours on GFP-PEST-expressing HT1080 acceptor cells. After incubation, GFP fluorescence quantification by FACS indicates that Killer EVs efficiently impair protein synthesis in acceptor cells. (D) Quantification of data from panel C. indicates that co-expression of VSV-G with Palm-DTA significantly improves Palm-DTA-containing EVs activity both at the level of protein synthesis inhibition (GFP MFI) and at the level of cell death induction (cell number). € A similar experiment as in C and D indicates that the effect of Killer EVs is dose-dependent as protein synthesis inhibition increases with increasing dose of EVs. Killer EVs are 5 times more efficient than Palm-DTA EVs. (F) Microscopic observation of GFP-PEST-expressing HT1080 acceptor incubated with Killer EVs indicates a total loss of cells after 3 days.

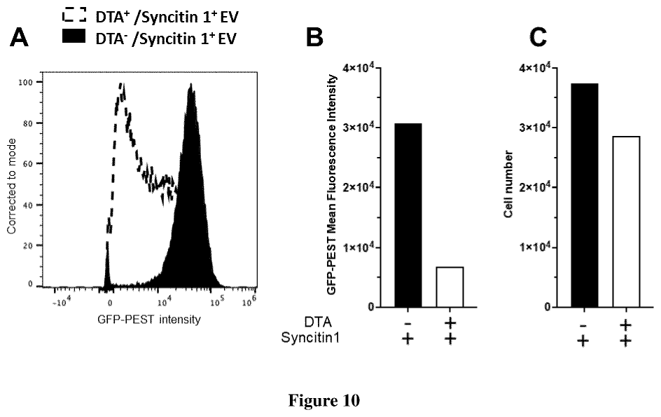

FIG. 10: Virus-free Killers EVs are potent in vitro
(A) DPH2KD donor cells expressing FKBP2-RFP-CD63, FRB-DTA-HA and Syncitin1, were treated or not with the dimerizer for 24h, prior isolating EVs. GFP-PEST-expressing HT1080 acceptor cells were treated or not with Syncitin1-positive EV loaded or not with DTA using the drug-inducible loading system. GFP fluorescence quantification by FACS indicates that virus-free Killer EVs (loaded with DTA and decorated with Syncitin1) efficiently impair protein synthesis in acceptor cells. (B-C). Quantification of data from panel A. indicates that Syncitin1⁺DTA⁺EVs show superior efficiency at the level of protein synthesis inhibition (GFP MFI) and at the level of cell death induction (cell number).

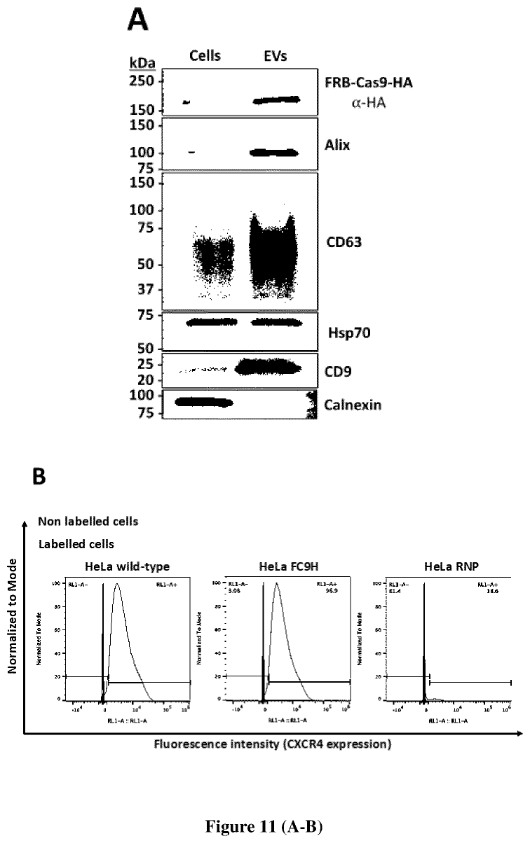

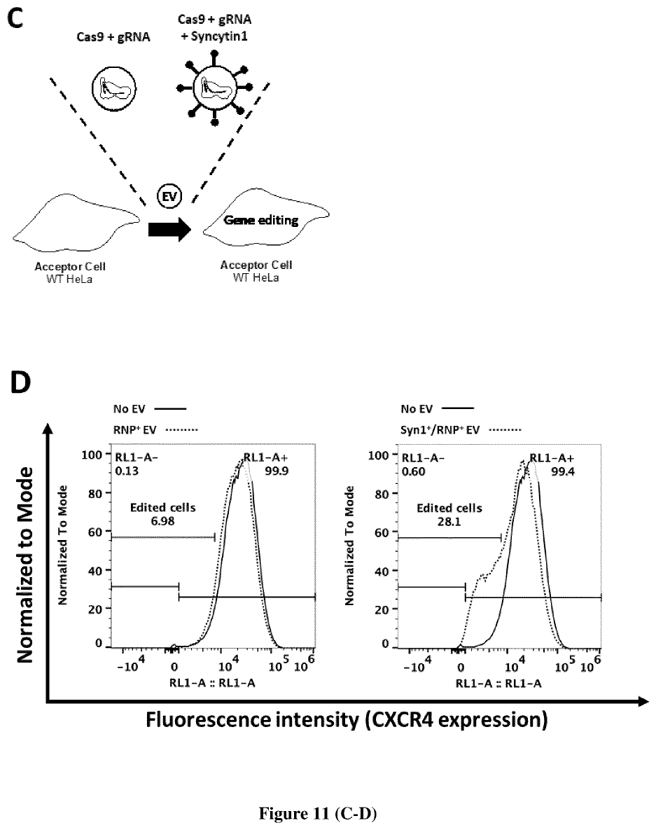

FIG. 11: Generation of editing EVs
(A) HeLa cells stably expressing FRB-Cas9-HA (FC9H) and CXCR4 gRNA and their derived EVs were characterized by western blot through different positive (Alix, CD63, Hsp70, CD9) and negative (Calnexin) markers. The expression of FRB-Cas9-HA was also analyzed using an antibody recognizing the HA-tag. Same amounts of proteins were loaded for both “Cells” and “EVs” conditions. (B) Wild-type HeLa, stable FC9H″ HeLa and stable RNP HeLa (FC9H/CXCR4 gRNA) were labelled (blue) or not (red) with the a-CXCR4 antibody coupled to APC, and analyzed by FACS using RLI laser. “Labelled cells” results were plotted against “Non labelled cells” results. The fluorescence intensity is plotted in x-axis, and the cell count is normalized to mode in y-axis. (C-D) Wild-type HeLa were incubated with EV carrying Cas9 and the gRNA only (RNP EVs), or additionally decorated with Syncytin-1 (Syn1/RNP EVs). After 48 hours, acceptor cells were collected and labelled with an α-CXCR4 antibody coupled to APC, and analyzed by FACS using RLI laser. “RNP+EVs” or “Syn1+/RNP+EVs” results were plotted against “No EV” condition. The fluorescence intensity is plotted in x-axis, and the cell count is normalized to mode in y-axis.

### EXAMPLE 1: METHODS FOR LOADING-FUSION EV SYSTEM

Cell culture. HeLa cells—wild type (from ATCC, Virginia, USA) and genetically modified—were grown in DMEM GlutaMAX (Gibco, Illinois, USA) supplemented with 10% FBS at 37° C. 5% CO₂. HeLa expressing NanoLuciferase-Hsp70 were generated according to Bonsergent et al. Nat Comm. 2021. HeLa CD8-GFP or FRB-NanoLuciferase-HA were selected with Hygromycin B (50 mg/mL, Invitrogen, Massachusetts, USA) after lipofectamine 2000 transfection. HeLa NLuc-CD63 were selected by Geneticin (50 mg/mL, Gibco, Illinois, USA) after lipofectamine 2000 transfection.

Transfection. Cells were transfected with Lipofectamine 2000 (Invitrogen) by mixing 10 μg of DNA to 10 μL of Lipofectamine 2000 in 2 mL total for a single 10 cm dish, and 1 μg of DNA to 1 μL of Lipofectamine 2000 in 100 μL total for a single 24 well-plate well during 20 minutes. Cells were incubated over 6 h at 37° C. 5% CO₂ with the transfection mix, and their media were replaced by a serum-free DMEM GlutaMAX (Gibco, Illinois, USA). The A/C Heterodimerizer drug (Takara Bio Inc., Shiga, Japan) was added at this stage for loading experiments.

EV isolation. Donor cells were transfected accordingly to the Transfection section. EVs were produced in serum-starvation in 5 mL of DMEM GlutaMAX per 10 cm dish. After 36h production, the media was recovered and centrifuged 20 min at 2,000 g 4° C. to remove dead cells and debris, then 30 min at 10,000 g 4° C. to remove large vesicles and apoptotic bodies (45Ti rotor, and then 1 h30 at 100,000 g 4° C. to isolate EVs (45Ti rotor, Optima™ XE-90 Ultracentrifuge, Beckman Coulter, California, USA). Finally, the 100 Kg pellet was recovered and re-centrifuged 1 h10 at 100,000 g 4° C. in PBS to wash out the media (SW55 rotor). The final pellet was resuspended into PBS and used immediately or stored at 4° C.

Floatation assay. An EV isolation was performed without the washing step. The 100 Kg pellet was resuspended into 1 mL 60% sucrose in PBS (prepared accordingly to MM Temoche-Diaz, Bio Protoc. 2020) and dropped in the bottom of a SW55 tube. One milliliter of 30% and then 1 mL of PBS were deposited above the 60% fraction. Samples were then centrifuged at least 15 h at 4° C. (SW55 rotor), and then recovered into 9 fractions of 300 μL. Luminescence activity of each fraction was directly analyzed. Each fraction was then diluted into 4 mL total PBS and centrifuged 1 h at 100,000 g 4° C. (MLA-50 rotor, Optima™ MAX-XP Ultracentrifuge, Beckman Coulter, California, USA) in order to wash out the sucrose and perform western blotting.

NLuc-based uptake assay and content delivery assay performed accordingly to Bonsergent et al. 2021 with EVs carrying FRB-NanoLuciferase-HA or NanoLuciferase-CD63 as donor EVs. The luminescence was read using Nano-Glo Luciferase Assay System (Promega, Wisconsin, USA) iD3 SpectraMax microplate reader (Molecular Devices, California, USA). Recruitment assay. Cells were seeded at DO on glass coverslips, and were co-transfected the next day with pC4-FKBP2-RFP-CD63 and pC4-FRB-NLuc-HA with the respective ratio 30%/70%. At day 3, the cells were treated or not with the A/C Heterodimerizer drug (Takara) during 1 h at 37° C., and then prepared for confocal microcopy observation by labelling FRB-NLuc-HA in green.

Cloning. PCR oligonucleotides were ordered to Eurofins Genomics (Luxembourg, Luxembourg). PCR reactions were performed according to Thermo Fisher or NEB protocols, digestion and ligation (vector: insert molar ratio of 1:3) according to NEB protocol and software. 2 μL of ligation product was used to transform 20 μL of competent bacteria (Library Efficiency™ DH5a Competent Cells, Thermo Fisher Scientific, Massachusetts, USA) at 42° C. 30 sec. Bacteria were recovered into 200 μL S.O.C. media during 1 h at 37° C. on agitation, and then spread and incubated on ampicillin or kanamycin agar plates over night at 37° C.

Plasmids. pC₄-GFP-HA was generated by Gregory Lavieu. VSV-G was purchased from AddGene (#8454). Syncytin-1 was given by Thierry Heidmann. pC₄-FRB-HA corresponds to pC₄-R_{H}E (ARIAD from Takara Bio). pC₄-FKBP₂-HA was generated by digesting pC₄-RHE and pC₄M-F2E (ARIAD) with XbaI and SpeI, and swapping FKBP₂ into empty pC₄-R_{H}E. pC₄-FKBP₂-RFP-CD63 was generated by amplifying RFP-CD63 (given by Walther Mothes), and inserting it into pC₄-FKBP₂-HA digested with EcoRI and BamHI. pC₄-FRB-NLuc-HA was generated amplifying NLuc (from NLuc-Hsp70, Bonsergent et al. 2021), and inserting it into pC₄-R_{H}E using SpeI restriction site.

Antibodies. Primary antibodies: Anti-TGN46 (PA5-23068, Invitrogen), Anti-hCD9 (Clone MM2-57, Millipore), Anti-hCD63 (556019, BD Pharmingen), Anti-HA (for IF, 66006-2-Ig, Proteintech; for WB, C29F4, Cell Signaling), Anti-Cherry (5993-100, BioVision), Anti-Calnexin (ab133615, Abcam), Anti-ALIX (Clone 3A9, 2171S, Cell Signaling), Anti-HSP70/HSP72 (Clone C92F3A-5, ADI-SPA-810F, Enzo Life Sciences), Anti-Actin (Clone C4, MAB1501, Millipore). Secondary antibodies for western blotting: Goat Anti-Rabbit IgG (H+L)-HRP Conjugate (1706515, Bio-Rad) and Goat Anti-Mouse IgG (H+L)-HRP Conjugate (1706516, Bio-Rad). Secondary antibody for immunofluorescence: Goat Anti-Mouse IgG (H+L) Highly Cross-Absorbed Secondary Antibody, Alexa Fluor™ 488 (A11029, Thermo Fisher Scientific).

Western blotting. Cells were collected and washed in PBS, the pellet was resuspended in lysis buffer (Tris 50 mM, NaCl 150 mM, Triton X-100 1%, protease/phosphatase inhibitor cocktail (Roche, Switzerland), pH 8) during 20 min on ice, and then centrifuged during 15 min at 20,000g to pellet the membranes and collect the supernatant. Cell lysates and EVs protein concentration were estimated using Micro-BCA™ Protein Assay Kit (Thermo Scientific, Illinois, USA). Samples were mixed with 4X Laemmli buffer (Bio-Rad, California, USA) completed with 10% B-mercaptoethanol (BME), except for CD63 protein which cannot be detected in presence of BME. Electrophoresis was performed on 4-20% polyacrylamide gels (Bio-Rad, California, USA) in Tris/Glycine/SDS Buffer (Bio-Rad), and proteins were transferred on Immun-Blot PVDF membranes (0.2 μm, Bio-Rad) using the TransBlot Turbo system (Bio-Rad). Precision Plus Protein™ Standards (Bio-Rad) was used as ladder. Membranes were then blocked into 0.05% Tween 5% milk in PBS during 1h at RT, and incubated overnight with the primary antibody diluted at 1/1000 in 0.05% Tween 5% milk in PBS. Membranes were then washed 1h in PBS 0.05% Tween, incubated with secondary antibodies diluted at 1/10,000 in PBS 0.05% Tween, and washed 1 h in PBS 0.05% Tween. Membranes were revealed using Clarity™ Western ECL Substrate (Bio-Rad) and ImageQuant™ LAS 400 (GE Healthcare Life Sciences, Chicago, USA). Image analysis and quantification were performed using Fiji software.

### Confocal Microscopy.

Cells were either seeded on glass coverslips 1 day before fixation if stable cell line, either seeded 2 days before and transfected the next day for transient protein expression. Cells were then washed out 3 times with cold PBS, incubated in 4% PFA 15 min at RT. If an antibody-labelling was performed, cells were then permeabilized with Triton-X100 (Sigma-Aldrich, Massachusetts, USA) 15 min at RT, incubated with primary antibody diluted at 1/500 2 h at RT, then with secondary antibody diluted at 1/2,000 1 h at RT, finally a DAPI staining was performed when needed at a 1/10,000 dilution. Coverslips were mounted with ProLong™ Diamond Antifade Mountant (Invitrogen).

Images were acquired using LSM 880 confocal microscope (ZEISS, Baden-Württemberg, Germany). Image analysis and quantification were performed using Fiji software.

Nanoparticles Tracking Analysis was performed using ZetaView x20 (Particle Metrix, Ammersee, Germany) with the following parameters: laser 488 nm, scatter, 11 positions, 1 cycle, sensitivity 80, shutter 100, pH7 entered, T° C. sensed. All samples were diluted into 1X filtered PBS.

### Example 2: Methods for “Killer-Evs”

Cell culture. HeLa and HT1080 cells (ATCC, Virginia, U.S.A.) and their transgenic derivatives were grown in DMEM medium (Gibco, Illinois, U.S.A.) complemented with 10% heat-inactivated Fetal Bovine Serum (Biowest, France) at 37° C. under 5% CO2 and high humidity. HT1080 cells medium was further complemented with MEM NEAA (Gibco, Illinois, U.S.A.).

Stable DPH2KD HeLa cells were obtained by lentiviral transduction of a shRNA targeting DPH2 (Horizon Discovery, Cat #VGH5518-200302258, U.K.) and selected with 4 μg/mL puromycin (Gibco, Illinois, U.S.A.). A stable GFP-PEST HT1080 clone was obtained by selecting cells with 0.5 mg/mL geneticin (Gibco, Illinois, U.S.A.) after transfection with a GFP-PEST encoding plasmid (Addgene, Cat #26821, Massachusetts, U.S.A.).

Transient transfections were performed using Lipofectamine 2000 (Invitrogen, Massachusetts, U.S.A.) according to the manufacturer's instructions.

Plasmid constructs. To construct the plasmid encoding DTA-HA, the sequence for DTA (obtained from Addgene, Cat #42521, Massachusetts, U.S.A.) was fused to the HA-tag sequence using the Infusion cloning strategy (Takara Bio Europe, France) with Xbal/Spel cloning sites into a pC4RHE backbone (ARIAD Pharmaceuticals, Massachusetts, U.S.A.). The DTA-HA construct was then subcloned into a pCDNA3.1 backbone (Invitrogen, Massachusetts, U.S.A.) using NheI/BamHI cloning sites.

To construct the plasmid encoding Palm-DTA-HA, the SNAP25 palmitoylation sequence (Greaves et al., JBC 2000) was inserted at the N-terminus of DTA-HA using Infusion cloning (Takara Bio Europe, France).

To construct the plasmid encoding FRB-DTA-HA, the FRB sequence was first cloned into a pcDNA3.1 backbone (Invitrogen, Massachusetts, U.S.A.) using the NheI/BamHI cloning sites and using plasmid pC4R_{H}E as an FRB template. Then, the DTA-HA sequence was cloned into BamHI/XbaI sites of this plasmid.

qRT-PCR. Total RNA was extracted from cells using the Nucleospin RNA kit (Macherey Nagel, France) according to the manufacturer's instructions. Equal amounts of total RNA were reverse transcribed using the iScript cDNA synthesis kit and subjected to qPCR using the iTaq SYBR green kit (Bio-Rad, France), all following the manufacturer's instructions. qPCR was performed in a CFX96 system (Bio-Rad, France) at 95° C. for 10 min, followed by 40 cycles at 95° C. for 15 sec, 60° C. for 30 sec, and 72° C. for 30 sec. DPH2 gene expression was normalized to the PGK housekeeping gene according to the 2-ΔΔCt formula.

Protein synthesis assay. Parental or DPH2KD Hela cells were seeded in 24 well plates before being co-transfected with plasmids encoding NanoLuc-Hsp70 and plasmids encoding either mock, or DTA-HA, or Palm-DTA-HA. 6 hours after transfection, cells were detached and split in triplicate wells of a 96 well plate. 24 hours later, cells were washed with DPBS and NanoLuc activity was measured in each well using the Nano-Glo Live Cell Assay System (Promega, Wisconsin, USA) following the manufacturer's instructions using the iD3 SpectraMax microplate reader (Molecular Devices, California, USA). The percentage of protein synthesis was calculated relative to the mock-transfected cells (mock set at 100%) for each cell type tested.

EV preparation. EV donor cells were transfected with the indicated plasmids for 16 hours before being incubated in serum-free DMEM for 24 hours. Conditioned medium was harvested and submitted to a 2000× g centrifugation for 20 min at 4° C. to remove cell debris, and then to a 100,000× g ultracentrifugation for 1 h 30 min at 4° C. (45 Ti rotor and Optima™ XE-90 Ultracentrifuge, Beckman Coulter, California, USA) to pellet EVs. The EV pellet was washed with DPBS and centrifuged 1 h 30 min at 100,000× g 4° C. (MLA 50 rotor with dedicated adaptors and Optima MAX-XP ultracentrifuge, Beckman Coulter, California, USA). The washed pellet was resuspended in DPBS and EVs were either stored at −20° C. (if destined to western blot analysis) or immediately applied on acceptor cells.

Western blot. Cells to be analyzed were scraped on ice in DPBS and pelleted at 1000× g for 5 min at 4° C. Cell pellets were resuspended in PBX lysis buffer (DPBS, Triton-X-100 1%, EDTA-free protease/phosphatase inhibitor cocktail (Roche, Switzerland)) and incubated on ice for 10 min with intermittent vortexing. Samples were then submitted to a 15,000× g centrifugation for 10 min at 4° C. to pellet nuclei and unbroken cells. Supernatants (cell lysates, CL) were collected. Protein concentration of cell lysate and EVs were obtained using the Micro BCA Protein Assay kit (Thermo Scientific, Illinois, USA). Samples were mixed with Laemmli buffer (Bio-Rad, France) containing 10% B-mercaptoethanol, except for CD63, and CD9 detection (no β-mercaptoethanol) and loaded on 4-15% polyacrylamide gels (Bio-Rad, France). After electrophoresis, proteins were transferred on PVDF membranes using the Trans-Blot Turbo system (Bio-Rad, France). Membranes were incubated with DPBS containing 0.05% Tween20 and 5% non-fat milk (blocking buffer), then with a 1/1000 dilution of primary antibody (α-Actin (Cat #MAB1501, Millipore, Germany), α-ALIX (Cat #2171, Cell Signaling, Massachusetts, U.S.A.), α-Calnexin (Cat #ab 133615, Abcam, U.K.), α-CD63 (Cat #556019, BD Bioscience, New Jersey, U.S.A.), α-CD9 (Cat #cbl162, Millipore, Germany), α-Hsp70 (Cat #ADI-SPA-810-D, Enzo LifeScience, New York, U.S.A.), α-HA (Cat #3724, Cell Signaling, Massachusetts, U.S.A.), α-mCherry (Cat #5993, BioVision, California, U.S.A.)) in blocking buffer overnight at 4° C. Membranes were then washed and finally incubated with a 1/5000 dilution of HRP-coupled secondary antibody (α-mouse or α-rabbit, Cat #115-035-003, Jackson ImmunoResearch, U.K.) in DPBS containing 0.05% Tween20. The HRP signal on membranes was developed using the Clarity Western ECL substrate (Bio-Rad, France) and imaged using the ImageQuant LAS 4000 (GE Healthcare Life Sciences, France).

Cytosol/membrane fractionation. Cells to be analyzed were scraped on ice in DPBS and pelleted at 1000× g for 5 min at 4° C. Cell pellets were resuspended in 5 volumes of a hypotonic lysis buffer (10 mM Tris-HCl pH 8, 0.5 mM MgCl₂ and EDTA-free protease/phosphatase inhibitor cocktail (Roche, Switzerland)) and incubated on ice for 10 min before being homogenized with 10 up-and-down passages through a 26 g needle. Tonicity was restored by the addition of 0.25 volume of the hypotonic buffer containing 0,6 M NaCl. Nuclei and unbroken cells were pelleted at 500× g for 5 min at 4° C. EDTA was added to the supernatant to a final concentration of 0.05 M before subjecting the samples to ultracentrifugation at 100,000× g for 30 min at 4° C. (MLA 50 rotor with dedicated adaptors and Optima MAX-XP ultracentrifuge, Beckman Coulter, California, USA). The resulting supernatant constituted the cytosolic fraction. The pellet was resuspended in PBX and centrifuged at 10,000× g for 15 min at 4° C. to pellet insoluble material. The supernatant constituted the membrane fraction.

Particle metrics. Nanoparticle Tracking Analysis was performed using the ZetaView® QUATT (Particle Metrix, Meerbusch, Germany) and its corresponding software (Zeta View 8.02.28). For the size measurements, the 448 nm laser in scatter mode was used. 1 ml of sample, diluted in DPBS, was loaded into the cell, and the instrument measured each sample at 11 different positions throughout the cell. After automated analysis of all positions and removal of any outlier positions, the mean, median, and mode (indicated as diameter) sizes were calculated by the optimized machine software.

FACS analysis. After treatments, cells were detached from cell culture plates with 0.05% Trypsin-EDTA and washed once in DPBS. Cells were finally resuspended in DPBS and kept on ice (less than one hour) until analyzed on an Attune NxT flow cytometer (Thermo Scientific, Illinois, USA). Each sample was incubated with 10 μg/mL DAPI (Merck Millipore, Massachusetts, U.S.A.) right before analysis. The gating strategy is depicted in Figure SIC. Data was analyzed using the FlowJo software (BD Bioscience, New Jersey, U.S.A.).

Microscopy. Live cells were visualized under an EVOS M5000 microscope at 20× magnification. Image analysis was performed using the ImageJ software (NIH, Maryland, U.S.A.).

### Example 3: Results

We developed a novel method to control the loading of a cargo into EVs on demand. These EVs are equipped, if necessary, with non-viral fusogen, therefore enhancing EV-cargo delivery into acceptor cells.

To acutely measure this process, we follow the fate of a luciferase-tagged cargo. Cargo loading was enabled through a drug-reversible inducible dimerization system. Briefly, donor cells were transfected with plasmids encoding for FKBP-tagged CD63, a classical membrane EV marker, and FRB-Nanoluciferase (NLuc) that is normally cytosolic. Upon addition of the dimerizing drug, FRB-Nluc interacts with FKBP-CD63 and is recruited into secreted EVs. This is accompanied by an enhanced delivery into acceptor cells. This phenomenon can be further enhanced when EVs are equipped with syncitin1, a mammalian fusogenic protein that trigger fusion between EV membrane and the plasma membrane of acceptor cells.

We anticipate that the first application will be the development of “editing EVs” that will deliver the cas9 editing machinery to cells/tissues of interest. Indeed, editing EVs that contain cas9 and a guide RNA against CxCR4, a plasma membrane-localized receptor and that are decorated with Syncitin1 increase the delivery capacity. Thus, Syn1+editing EVs can efficiently knock out CxCr4 within approximatively 30% of the acceptor cells (FIG. 11). Another application will be the delivery of toxin through “Killer EVs”, aiming at the specific ablation of cells/tissues, including tumors.

Using our novel process, we demonstrated here that the catalytic domain of the Diphteria toxin (DTA), that is responsible for protein synthesis inhibition and ultimately cell death, can be delivered to acceptor cells via functionalized EVs. This led to protein synthesis inhibition and death of acceptor cells.

This novel method and the derived applications promise to open new doors in precision care medicine, especially when EVs will be equipped with antibodies raised against cell specific antigens.

### REFERENCES

Throughout this application, various references describe the state of the art to which this invention pertains. The disclosures of these references are hereby incorporated by reference into the present disclosure.

1. An isolated extracellular vesicle (EV) functionalized with an ERV syncytin and loaded with one or more cargo(s), and that is optionally functionalized with a targeting moiety.

2. The isolated EV of claim 1 wherein the ERV syncytin is selected from the group consisting of a human syncytin, a murine syncytin, syncytin-Ory1, syncytin-Car1, syncytin-Rum1 or their functional orthologs.

3. The isolated EV of claim 1 wherein the ERV syncytin is a syncytin-1 polypeptide that comprises the amino acid sequence as set forth in SEQ ID NO: 2 (SDGGGX2DX2R) and is capable of binding to the ASCT1 receptor.

4. The isolated EV of claim 3 wherein the syncytin-1 polypeptide comprises the amino acid sequence as set forth in SEQ ID NO: 3 (SDGGGVQDQAR).

5. The isolated EV of claim 4 wherein the syncintin-1 polypeptide comprises an amino acid sequence having at least 70% of identity with the amino acid sequence that ranges from the amino acid residue at position 21 to the amino acid residue at position 538 in SEQ ID NO: 1.

6. The isolated EV of claim 1 wherein the one or more cargo(s) is selected from the group consisting of an organic molecule, a polymer, a polypeptide, a polynucleotide and a small organic compounds having a molecular weight of more than 50 and less than 2,500 daltons.

7. The isolated EV of claim 6 wherein the one or more cargo(s) is a polynucleotide.

8. The isolated EV of claim 6 wherein the one or more cargo(s) is a polypeptide selected from the group consisting of a DNA targeting endonucleases selected from the group consisting of Transcription Activator-Like Effector Nucleases (TALENs), Zinc-Finger Nucleases (ZFNs), CRISPR-associated endonucleases, base-editing enzymes, and prime editors.

9. The isolated EV of claim 1 wherein the one or more cargo(s) is a toxin.

10. The isolated EV of claim 9 wherein the toxin is a diphtheria toxin or a toxic fragment thereof.

11. The isolated EV of claim 10 wherein the diphtheria toxin comprises the residues 1-389 of SEQ ID NO: 4.

12. The isolated EV of claim 6 further comprising a structural polypeptide that forms a dimer with the polypeptide.

13. The isolated EV of claim 12 wherein the structural polypeptide and the polypeptide are fused either directly or via a linker to respective domains that are capable of dimerization in the presence of a compound.

14. The isolated EV of claim 13 wherein the structural polypeptide is fused to an FKBP domain and the polypeptide is fused to an FRB domain, or the structural polypeptide is fused to the FRB domain and the polypeptide is fused to the FKBP domain, whereby it is possible to dimerize the FKBP domain and the FRB domain in the presence of rapamycin during production of an EVs.

15. The isolated EV of claim 14 further comprising a loading system wherein a transmembrane protein is fused to a FKBP2 domain.

16. The isolated EV of claim 15 wherein the transmembrane protein is a tetraspanin.

17. The isolated EV of claim 16 wherein the tetraspanin is CD63.

18. The isolated EV of claim 15 wherein the loading system comprises the amino acid sequence as set forth in SEQ ID NO: 7.

19. (canceled)

20. A method of treating cancer in a subject in need thereof, comprising administering to the subject a therapeutically effective amount of the isolated EV according to claim 1

21. A pharmaceutical composition that comprises an amount of the isolated EVs according to claim 1.


SEQUENCE LISTING (7 sequences)

SEQ ID NO: 1 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 2 - protein, 9 aa, synthetic construct
SDGGGXDXR

SEQ ID NO: 3 - protein, 11 aa, Homo sapiens
SDGGGVQDQAR

SEQ ID NO: 4 - protein, 507 aa, Diphthera sp.
DSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 5 - protein, 216 aa, synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 6 - protein, 93 aa, synthetic construct
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 7 - protein, 694 aa, synthetic construct
MASRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETRMASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGALYKSGLRSRAMAVEGGMKCVKFLLYVLLLAFCACAVGLIAVGVGAQLVLSQTIIQGATPGSLLPVVIIAVGVFLFLVAFVGCCGACKENYCLMITFAIFLSLIMVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM